(19) United States
(12) Patent Application Publication
(10) Pub. No.: US 2003/0219899 A1
(43) Pub. Date: Nov. 27, 2003

(54) Mosaic adenoviral vectors

(76) Inventor: Nikolay Korokhov, Birmingham, AL (US)

Correspondence Address: Benjamin Aaron Adler, ADLER & ASSOCIATES, 8011 Candle Lane, Houston, TX, 77071, US

(21) Appl. No.: 10/372,615

(22) Filed: Feb. 21, 2003

Related U.S. Application Data
(63) Continuation-in-part of application No. 10/124,796, filed on Apr. 17, 2002.
(60) Provisional application No. 60/284,331, filed on Apr. 17, 2001.

(51) Int. Cl. C12N 15/861; C12N 7/00

(52) U.S. Cl. 435/456; 435/235.1

(57) ABSTRACT

The present invention provides adenoviral vectors (Ad) that incorporate multiple distinct capsid modifications in a single virus particle, resulting in Ad that have improved gene delivery capacities and/or vector function. The capsid modifications include (a) addition of a heterologous targeting ligand; (b) a fiber shaft with altered length; (c) capsid modification that alters the charge of the adenoviral vector; and (d) replacing the capsid proteins with capsid proteins from another serotype.

### CROSS-REFERENCE TO RELATED APPLICATION

This is a continuation-in-part application of application U.S. Ser. No. 10/124,796, filed Apr. 17, 2002, which claims benefit of provisional patent application U.S. Ser. No. 60/284,331, filed Apr. 17, 2001, now abandoned.

### BACKGROUND OF THE INVENTION

1. Field of the Invention

The present invention relates generally to the field of adenovirus vectors. More specifically, the present invention relates to adenoviral vectors that incorporate multiple distinct capsid modifications.

2. Description of the Related Art

The human adenoviruses of serotype 5 (Ad5) is the most commonly used vector for gene therapy applications. Its utility as a gene delivery vehicle is largely based on its ability to infect a wide range of cell types with a remarkable efficiency (Russel, 2000). There are, however, some limiting features for the use of Ad5 vectors for gene therapy. First of all, the widespread distribution of the adenoviral primary receptor—the coxsackie-adenovirus receptor (CAR)—precludes specific gene delivery to target cells. Furthermore, often the very cell types that are to be targeted, such as tumor cells, lack CAR and are therefore not permissive for infection by non-targeted adenovirus (Russel, 2000; Pickles et al., 1998). Thus, to achieve the levels of efficiency required in the context of cancer gene therapy, it may be necessary to route the adenovirus via CAR-independent pathways. Further, to achieve the optimal therapeutic index, such CAR-independent gene transfer would optimally be achieved via tumor-specific cellular markers.

Viral Vectors can be Modified to Effect Cell-Specific Gene Transfer

Based on the above considerations, it is hypothesized that modifications of adenoviral tropism can accomplish enhancement of tumor cell transduction. Precedents for modifying viral tropism to accomplish enhanced and/or cell-specific gene delivery have been established using retroviral vectors. A number of studies have shown that retroviral cell-binding activity or tropism can be altered by modifications of the viral envelope glycoproteins that interact with specific receptors on the cell surface. One approach involved the construction of “pseudotypes” in which the retroviral genome is coated by the envelope protein of another virus. The host range of the pseudotyped particle is thus dictated by the virus providing the envelope protein.

Other strategies for achieving cell specific targeting of retroviral vectors involved specific genetic modification of the envelope. This has been accomplished by fusing to the retroviral envelope protein either (a) an antibody fragment which recognizes a cell specific antigen, or (b) a ligand with a cognate receptor on the target tissues. The demonstration of infection of human cells through the targeted receptor indicates that it is possible to employ genetic methods to engineer recombinant viral vectors with modified tropism. These studies demonstrate the capacity to achieve enhanced and cell-specific gene delivery by modifications of viral tropism.

Strategies to Alter Adenoviral Tropism are Based Upon an Understanding of Viral Entry Mechanisms.

Consideration of these studies designed to modify retroviral tropism has established the concept that cell-specific transduction may be accomplished with genetic modifications of viral domains dictating entry. It should thus be possible to achieve a similar end with adenoviral vectors based upon consideration of adenoviral entry dynamics. Following administration of the adenovirus vector, three distinct sequential steps are required for expression of the therapeutic gene in target cells: (i) attachment of the adenovirus to specific receptors on the surface of the target cell; (ii) internalization of the virus; and (iii) transfer of the gene to the nucleus where it can be expressed. Thus, any attempt to modify the tropism of an adenovirus vector must preserve its ability to perform these three functions efficiently. Furthermore, the modification of adenovirus tropism must be approached with knowledge of the biology of adenovirus infection.

In this regard, it has been shown that the globular carboxy-terminal “knob” domain of the adenovirus fiber protein is the ligand for attachment to the adenovirus cellular receptor, the first step in infection. A trimeric fiber protein attaches non-covalently to the penton base and protrudes from each of the 12 vertices of the icosahedral viral particle. The amino-terminal fiber tail is separated from the knob domain by a long rod-like shaft comprising a 15-amino acid residue motif repeated twenty-two times in human adenovirus types 2 and 5. It has been demonstrated that adenovirus infection can be blocked both by a recombinant adenovirus type 5 (Ad5) trimeric knob expressed in E. coli, or by an anti-knob antibody. Thus, the knob is both necessary and sufficient for virion binding to host cells.

Following attachment, the next step in adenovirus infection is internalization of the virion by receptor-mediated endocytosis. This process is mediated by the interaction of Arg-Gly-Asp (RGD) sequences in the penton base with secondary host cell receptors, the integrins α_{V}β₃ and α_{V}β₅. Post-internalization, the virus is localized within the cellular vesicle system, initially in clathrin-coated pits and then in cell endosomes. Acidification of the endosomes allows the virions to escape and enter the cytosol. This step has been hypothesized to occur via a pH-induced conformational change that causes an alteration in the hydrophobicity of the adenoviral capsid proteins, specifically at the penton base that allows its interaction with the vesicle membrane. Upon capsid disassembly and cytoplasmic transport, the viral DNA localizes to the nuclear pore and is translocated to the nucleus of the host cell. This understanding of the adenovirus entry pathway should facilitate attempts to modify the tropism of adenoviral vectors to permit the targeting of specific cell types.

Tropism-Modified Adenoviral Vectors for Gene Delivery

There have been various attempts to modify viral tropism with the ultimate intention to achieve both more efficient and more specific infection to target tissues and cells (Russel, 2000). For example, strategies have been endeavored to modify the native trophism of adenovirus to allow CAR-independent infection. Such CAR-independence of target cell binding/entry predicates increased gene transfer efficiency. A variety of strategies have been proposed to achieve adenovirus trophism modification including the employment of heterologous molecules, termed “re-targeting complexes”, which cross-link the adenovirus to non-CAR receptors. In addition, genetic modifications of the adenovirus capsid have been shown to accomplish the same end. In both instances, initial anchoring of the adenovirus to a non-native receptor is not inconsistent with target cell binding/entry followed by effective gene delivery. Indeed, it has been shown that it is possible to route adenovirus via a wide variety of heterologous cellular pathways. In many of these instances, retargeted entry can allow dramatic enhancements of adenovirus gene transfer efficiency via the circumvention of target cell CAR deficiency.

For practical gene therapy applications, the genetic capsid modification approach to trophism modification offers several advantages. This approach allows the achievement of CAR-independent gene delivery via diverse mechanisms. Heterologous targeting peptides have been incorporated into the HI loop (Dmitriev et al., 1998; Krasnykh et al., 1998; Xia et al., 2000) and COOH terminus (Michael et al., 1995; Wickham et al., 1996, 1997; Yoshida et al., 1998) of the fiber protein, the penton base, hexon, and the minor capsid proteins, pIIIa and pIX. In addition, it has been shown that selected adenovirus serotypes achieve entry via distinct receptors different from that used by serotype 5, the serotype of the widely used adenoviral vector. On this basis, serotype chimerism for the fiber knob or for the entire fiber has allowed routing of the virus into non-CAR pathways.

It is noteworthy that in vivo gene delivery may be affected by factors over-and-above target cell adenovirus receptor levels. Specifically, the ability of adenovirus particles to transit in the context of anatomic barriers can affect in vivo efficacy. Thus, modulating the length of the fiber shaft, a maneuver which effects particle size, and thus, its distribution physiology, has resulted in altered in vivo gene delivery profiles. Moreover, genetic capsid alterations to modify particle charge may affect in vivo gene delivery dynamics. Therefore, these distinct strategies—incorporation of heterologous targeting peptides, capsid protein chimerism, fiber shaft modulation, and capsid charge modulation—can allow trophism alteration of adenovirus with the achievement of improved gene delivery dynamics.

Although the modifications in the adenoviral capsid mentioned above can achieve corresponding alteration in trophism, it has not been shown such alterations may be achieved in combination, resulting in additive or synergistic improvements in gene delivery and/or vector function. Thus, the prior art is deficient in adenoviral vectors that incorporate multiple distinct capsid modifications to achieve altered trophism and enhanced gene delivery capacities. The present invention fulfills this long-standing need and desire in the art.

### SUMMARY OF THE INVENTION

The present invention provides adenoviral vectors (Ad) that incorporate multiple distinct capsid modifications such as incorporation of heterologous targeting ligand, capsid protein chimerism, fiber shaft modulation and capsid charge modulation. The resulting adenoviral vector would have improved gene delivery capacities and/or vector function.

In one aspect of the present invention, there is provided an adenoviral vector comprising one or more heterologous targeting ligands incorporated into more than one capsid proteins. The capsid protein can be a hexon, the knob domain of fiber protien, p3 protein, p9 protein and/or penton. In one embodiment, the heterologous targeting ligand is incorporated into the hexon and the knob domain of fiber protein. In general, the targeting ligands are physiologic peptide ligands, phase displayed peptide ligands, single chain antibodies (scFv) or components of single chain antibodies such as V_{H} and CDR3 regions of the single chain antibody.

In another aspect of the present invention, there is provided an adenoviral vector comprising one or more capsid proteins modified by replacement with capsid proteins from another serotype. In general, the capsid proteins can be hexon, knob domain of fiber protein, p3 protein, p9 protein and/or penton. In one embodiment, the fiber knob of adenovirus serotype 5 is replaced by the fiber knob of adenovirus serotype 3 or 37. The modified adenoviral vector can further comprises a heterologous targeting ligand. For example, there is provided an adenoviral vector wherein the knob domain of fiber protein is replaced by knob domain from another serotype and the heterologous targeting ligand is incorporated in the knob domain.

The present invention also provides an adenoviral vector comprising capsid proteins derived from multiple distinct serotypes. In one embodiment, the adenoviral vector expresses both the fiber knob of adenovirus serotype 5 and adenovirus serotype 3.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows the design and analysis of a modified Ad3 vectors. FIG. 1A is a map of Ad5.F5/3.Ct.His, showing the localization of a short peptide linker (P(SA)₄P) and a six-His containing peptide (RGDSH₆) on the carboxy-terminus of the Ad3 fiber knob. The GFP and LUC expression cassettes are also indicated. Vector Ad5.F5/3 is essentially the same, except that it lacks the sequence encoding the peptide addition. FIG. 1B shows the confirmation of fiber region of the viral genomes by PCR. PCR 1 resulted in expected amplification products of 756 bp (lane 1) and 813 bp (lane 2) for Ad5.F5/3 and Ad5.F5/3.Ct.His respectively. PCR 2 resulted in amplification products of 138 bp (lane 1) and 195 bp (lane 2) for Ad5.F5/3 and Ad5.F5/3.Ct.His respectively. Lane M: 1 kb ladder.

FIG. 2 shows Western blot analysis of the fiber proteins of denatured Ad5.F5/3 (lane 1) and Ad5.F5/3.Ct.His (lane 2). FIG. 2A shows verification of fiber lengths by detection with anti-Ad5 fiber tail mAb 4D2. The fibers of Ad5.F5/3.Ct.His are of expected length, i.e. slightly larger than the fibers of Ad5.F5/3. FIG. 2B shows verification of presence of the His tag on the fibers of Ad5.F5/3.Ct.His by detection with anti-five-His monoclonal antibody. Size markers are indicated in kDa.

FIG. 3 shows binding of anti-five-His monoclonal antibody to Ad5.F5/3.Ct.His, but not to Ad5.F5/3, thus demonstrating the accessibility of the His tag on viral particles of Ad5.F5/3.Ct.His. A dilution range of virus immobilized in the wells of an ELISA plate was incubated with anti-five-His mAb and subsequently with an alkaline phosphatase conjugate for detection. Results are the mean of triplicate experiments.

FIG. 4 shows dose dependent inhibition by imidazole of Ad5.F5/3.Ct.His-mediated, but not Ad5.F5/3-mediated, gene transfer to U118MG-HissFv.rec cells, demonstrating that Ad5.F5/3.Ct.His is capable of mediating gene transfer via specific interaction between the His tag and the artificial His-tag receptor. Prior to infection for 30 min with the respective virus (MOI=100 virus particles per cell), the U118MG-HissFv.rec cells expressing AR were incubated for 10 min at room temperature with 0, 2.5 or 25 mM imidazole in PBS. Luciferase activities detected in the lysates of infected cells 24 hours post-infection are given as percentages of the activity in the absence of imidazole. Results are the mean of quadruplicate experiments.

FIG. 5 shows infection of HUVEC cells with Ad vectors containing chimeric fiber proteins. HUVECs grown in the wells of 24-well plates to 80-90% confluence were infected with either Ad5/3LucI (gray bars), Ad5/37Luc1 (black bars) or Ad5LucI (white bars) at mulltiplicities of infection (MO1) of 100 or 1000 viral particles per cell. Recombinant AD5 fiber knob expressed in E. coli was employed at 50 ug/ml as a blocker of CAR. The cells were collected twenty four hours post-infection, lysed and the luciferase activity in the lysates was measured in relative light units (RLU).

FIG. 6 shows the strategy for mosaic virus generation. The virus genome of Ad5 and Ad5/3 based vectors were transferred into 293 cells by co-infection of both viruses. The cells infected by both viruses express two types of fibers, the Ad5 wild-type fiber and the Ad5/3 chimeric fiber. Both types of fibers assemble on the virion at random to generate the mosaic viruses.

FIG. 7 shows mosaic virus generation by co-infection of Ad5 and Ad5/3 at various ratios. HeLa cells were infected by crude virus lysate of Ad5luc1, Ad5/3luc1, mixtures of Ad5luc1+Ad5/3luc1 at various ratios (open bar) or four kinds of mosaic viruses, AdMSluc1 (closed bar). Luciferase activity was measured after 48hr incubation and is expressed as relative light units (RLU) per mg protein. Each point represents the mean of three experiments +/−SD.

FIG. 8 shows amplification of viral DNA by PCR. DNA extracted from 1×10⁸ viral particles of each virus was amplified by PCR with primer pairs corresponding to major late promoter region, Ad5 knob region and Ad3 knob region. Each region was amplified in 25 cycles or 28 cycles of PCR. The template DNA was extracted from Ad5luc1 (lane 1), a mixture of 70% Ad5luc1+30% Ad5/3luc1 (lane 2), AdMSluc1 (lane 3) or Ad5/3luc1 (lane 4). All PCR products showed the correct size fragment confirmed by size marker (lane M). PCR products from each backbone plasmid serve as positive control (lane C).

FIG. 9 shows Western blotting for fiber protein. Viral particles (2×10⁸) of each purified virus sample were dissociated in SDS sample buffer and heated at 96° C. for 3 min. Viruses bound to immobilized anti-Ad5 knob mAb 1D6.14 on a 96-well plate were also solubilized in SDS buffer after the binding reaction. All samples were separated on a 4 to 12% gradient polyacrylamide gel followed by electrotransfer onto PVDF membrane. After being blocked with TBST-casein, the membrane was treated with mAb 4D2 followed by treatment with goat anti-mouse IgG-HRP conjugate. The color reaction was developed by incubation of the membrane with Sigma Fast diaminobenzidine. Lanes 1 & 2, Ad5luc1; lanes 3 & 4, Ad5luc1+Ad5/3luc1; lanes 5 & 6, AdMSluc1; lanes 7 & 8, Ad5/3luc1. Samples in lanes 2, 4, 6 and 8 were first bound to immobilized mAb 1D6.14.

FIG. 10 shows infectivity profile of Ad5, Ad5/3 and mosaic virus in various cell lines. Ovarian cancer cells SKOV3.ip1 and OV-4, pancreas cancer cells Panc-I, lung cancer cells NCI-H157 and NCI-H358, uterine cervical cancer cells HeLa and C33A, glioma cells U118 and 293HEK cells were infected by Ad5luc1 (open bar), Ad5/3luc1 (closed bar), a mixture of Ad5luc1+Ad5/3luc1 (gray bar) or AdMSluc1 (checked bar) at 100 viral particles/cell. Luciferase activity was measured after 48 hr incubation and expressed as relative light units (RLU). Each point represents the mean of three experiments +/−SD.

FIG. 11 shows FACS analysis for CAR expression in tumor cell lines. Two hundred thousand SKOV3.ip1, HeLa or C33A cells were incubated with primary mAb RmcB to human CAR. The cells were then washed with 1 ml of PBS/BSA/azide and incubated with the secondary FITC-labeled goat anti-mouse IgG. Then 1×10⁴ cells were analyzed by flow cytometry. Expression levels of CAR and negative control were shown as areas filled in gray and black, respectively.

FIG. 12 shows mosaic virus infectivity in the presence of purified Ad3 and/or Ad5 knob protein. One hundred thousand C33A cells (panel A) or SKOV3.ip1 cells (panel B) were preincubated with the indicated concentrations of each recombinant knob protein for 10 min followed by the AdMSluc1 infection at 100 viral particle/cell. The knob proteins used for competition were as follows: Ad3 knob (closed square), Ad5 knob (closed diamond) and Ad3+Ad5 knob (closed triangle). Luciferase assays were performed after 48 hr incubation, and the luciferase activity was expressed as relative light units (RLU). Each point represents the mean of three experiments +/−SD.

FIG. 13 shows infectivity of Ad5 and Ad5/3 in the presence of purified Ad3 or Ad5 knob protein. One hundred thousand HeLa cells were preincubated with the indicated concentrations of purified Ad5 knob protein (panel A) or purified Ad3 knob protein (panel B) for 10 min followed by Ad5luc1 (closed diamond) or Ad5/3luc1 (closed square) infection at 100 viral particles/cell. Luciferase assay was performed after 48 hr incubation, and the luciferase activity was expressed as relative light units (RLU). Each point represents the mean of three experiment +/− SD.

FIG. 14 shows correlation between mosaic virus infectivity and CAR expression level. One hundred thousand parental U118 cells (open bar), modified U118-hCAR cells (gray bar) and U118-hCAR-tailless cells (closed bar) were infected by Ad5luc1, Ad5/3luc1 and AdMSluc1 at 100 viral particles/cell. Luciferase assay was performed after 48 hr incubation. Each luciferase activity with Ad5luc1 or AdMSluc1 was expressed as the ratio to the Ad5/3 luciferase activity.

FIG. 15 shows gene transfer to human heart tissue samples in vitro by Ad5Luc1 or Ad5lucF_{HI}RGD. Each bar represents a mean of six replicates (mean+_S.D.).

FIG. 16 shows gene transfer to human heart tissue samples in vitro by Ad5Luc1, Ad5lucF_{HI}RGD, Ad5/3Luc1 or Ad5/37Luc1. Each bar represents a mean of six replicates (mean±S.D.).

### DETAILED DESCRIPTION OF THE INVENTION

An aspect of the present invention is to develop adenoviral vectors based upon the complex mosaic paradigm to enhance gene delivery to target cells. The present invention presents a novel paradigm of adenoviral trophism modification based on simultaneous incorporation of multiple distinct capsid modifications. This “complex mosaic” strategy would exploit the benefits of the various component modification strategies in the context of a single vector particle, which thus embodies the advantages of the contributing alterations. In addition to additive effects, various possibilities for functional synergy may also accrue in this general approach.

Genetic capsid modifications may allow tropism-modification of Ad with improvements in infectivity via the achievement of CAR-independent cellular entry. Two distinct mechanisms to achieve this end have been reported: (i) exploiting cellular integrins via genetic incorporation of heterologous integrin-binding peptides into the Ad capsid; and (ii) exploiting non-serotype 5 Ad receptors via genetic fiber chimeras. Specifically, serotype 5 Ad vectors which incorporate the knob domains of serotype 3 adenovirus, as well as Ad vectors that incorporate the integrin-binding peptide RGD4C at the HI loop of the serotype 5 knob have been constructed. Each of these approaches achieves CAR-independent gene delivery to target cells. Such CAR-independent gene delivery has enhanced overall gene transfer efficacy in both instances.

It is logical to speculate that the distinct mechanisms of CAR-independent cellular entry may function in an additive manner. Thus, simultaneous employment of both integrin-binding and non-serotype 5 receptor binding may allow an additive effect with respect to Ad entry. Evaluation of this hypothesis requires the derivation of a series of Ad vectors possessing both knob serotype chimerism and RGD4C insertions within their respective fiber knob HI loops. In this regard, the utility of the HI loop on non-5 serotype Ad vectors has not been explored before and thus this set of studies represents a novel direction within the field.

It is also logical to speculate that targeting ligands will perform differentially in the context of distinct capsid locales. Although the integrin binding peptide RGD4C has been incorporated at the HI loop of the Ad5 fiber knob, previous investigators have identified alternative locales within the capsid for such incorporations. On this basis, the present invention will evaluate the optimal locale for incorporation of a given peptide for infectivity enhancement of Ad. The instant invention will also evaluate the potential of a given targeting ligand to further increase infectivity when simultaneously incorporated at more than one site within the capsid. Such direct and rigorous comparisons of ligands within alternative capsid sites and the testing of “complex mosaics” Ad vectors consisting of ligand incorporations at various sites have not been reported before within the field. Data disclosed in the instant invention will provide new and fundamental information with respect to the key biologic parameters predicating optimal Ad infectivity. Data from these studies will also provide a database allowing the derivation of an Ad vector with an optimized capacity to achieve CAR-independent cellular entry/gene delivery.

Moreover, the present invention will examine adenoviral capsid alterations that may affect gene transfer efficiency by means other than altered target cell receptor recognition. Altered particle size or charge can affect interaction with anatomic barriers, and thus alter in vivo delivery efficiency. It is thus clear that genetic capsid modifications involving various distinct alterations of adenoviral biology such as incorporation of heterologous targeting peptides, capsid protein chimerism, fiber shaft modulation, and capsid charge modulation may be used to enhance in vivo adenovirus gene transfer efficiency.

Thus, an object of the present invention is to demonstrate the feasibility of incorporating multiple distinct capsid modifications within a single vector, termed “complex mosaic” particle, that would provide a basis of improved gene delivery capacities/vector function compared to an adenovirus which is altered on a single capsid site. These mosaic designs may include, but are not limited to, the following modifications: (1) serotype chimerism and incorporation of heterologous ligand; (2) serotype chimerism of more that one capsid protein; (3) incorporation of heterologous ligands at more that one capsid focus; (4) altered fiber shaft length in combination with any, or all, of the above; and (5) alterations specifically designed to modify the charge of adenovirus, in combination with any or all, of the above.

As used herein, the terms “serotype chimerism” refers to a virus with capsid proteins derived from multiple distinct serotypes.

As used herein, the term “capsid protein chimerism” refers to a capsid protein containing components derived from multiple distinct serotypes.

As used herein, the terms “knob serotype chimerism” refers to a virus with fiber knobs derived from multiple distinct serotypes.

As used herein, the terms “heterologous targeting ligand” refers to a binding moiety that can attach the virus to non-native receptor.

In summary, the present invention provides an adenoviral vector comprising one or more of the following modifications: a) addition of heterologous targeting ligand; b) a fiber shaft with altered length; c) capsid modification that results in charge alteration of the adenoviral vector; and d) capsid protein modification by replacement with capsid protein from another serotype.

In one aspect of the present invention, there is provided an adenoviral vector comprising one or more heterologous targeting ligands incorporated into more than one capsid proteins. The capsid protein can be a hexon, the knob domain of fiber protien, p3 protein, p9 protein and/or penton. In one embodiment, the heterologous targeting ligand is incorporated into the hexon and the knob domain of fiber protein. The modified adenoviral vector can further comprises a fiber shaft with altered length. In general, the targeting ligands are physiologic peptide ligands, phase displayed peptide ligands, single chain antibodies (scFv) or components of single chain antibodies such as V_{H} and CDR3 regions of the single chain antibody. In one embodiment, the heterologous targeting ligand comprises the peptide sequence RGD.

In another aspect of the present invention, there is provided an adenoviral vector comprising one or more capsid proteins modified by replacement with capsid proteins from another serotype. In general, the capsid proteins can be hexon, knob domain of fiber protein, p3 protein, p9 protein and/or penton. In one embodiment, the fiber knob of adenovirus serotype 5 is replaced by the fiber knob of adenovirus serotype 3 or 37. The modified adenoviral vector can further comprises a fiber shaft with altered length. In another embodiment, the modified adenoviral vector further comprises a heterologous targeting ligand. For example, there is provided an adenoviral vector wherein the knob domain of fiber protein is replaced by knob domain from another serotype and the heterologous targeting ligand is incorporated in the knob domain.

The present invention also provides an adenoviral vector comprising capsid proteins derived from multiple distinct serotypes. Examples of capsid proteins are listed above. In one embodiment, the adenoviral vector expresses both the fiber knob of adenovirus serotype 5 and adenovirus serotype 3.

The present invention further provides an adenoviral vector which is charge-altered as a result of capsid modification, wherein said adenoviral vector also contains a modification such as incorporation of a heterologous targeting ligand, an altered fiber shaft length, or a capsid protein modified by replacement with capsid protein from another serotype.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Addition of Heterologous Ligand to the Adenovirus Serotype 3 Fiber Knob

The present example demonstrates that the carboxyl-terminus of Ad3 fiber knob, like the Ad5 fiber knob, has suitable sites for incorporation of heterologous ligands.

There is an increased interest in usage—for gene therapy purposes—of the adenovirus serotype 3 (Ad3) fiber knob, the structure of which only recently has been presented (Durmort et al., 2001). Adenovirus 3 is a non-CAR binding serotype of adenovirus with a tropism distinct from Ad5 (Defer et al., 1990; Krasnykh et al., 1996; Stevenson et al., 1995; Von Seggern et al., 2000). In general, adenoviral cell tropism is regarded to be largely dependent on the initial binding event of the adenoviral fiber knob domain to a cognate cellular receptor. In case of Ad5 this receptor is CAR; however, for Ad3 an as yet unknown cellular receptor exists (Defer et al., 1990; Stevenson et al., 1995; Von Seggern et al., 2000; Roelvink et al., 1998).

Several studies have demonstrated that adenovirus tropism can be modified by replacing the fiber, or the fiber knob region, by that of another adenovirus serotype (Krasnykh et al., 1996; Gall et al., 1996; Shayakhmetov et al., 2000; Zabner et al., 1999). In this regard, it was shown that Ad5 based vectors carrying the Ad3 fiber knob, exhibit an Ad3 type tropism (Krasnykh et al., 1996; Stevenson et al., 1997). It has become apparent that some clinically relevant tissues exhibit differential expression of Ad3 and Ad5 receptors (Stevenson et al., 1997). Moreover, several target cell lines have been identified to which Ad3 receptor-mediated infection was more efficient than CAR-mediated infection (Von Seggern et al., 2000; Stevenson et al., 1997; Su et al., 2001). On this basis, Ad3 tropism is also becoming of interest for gene therapy applications.

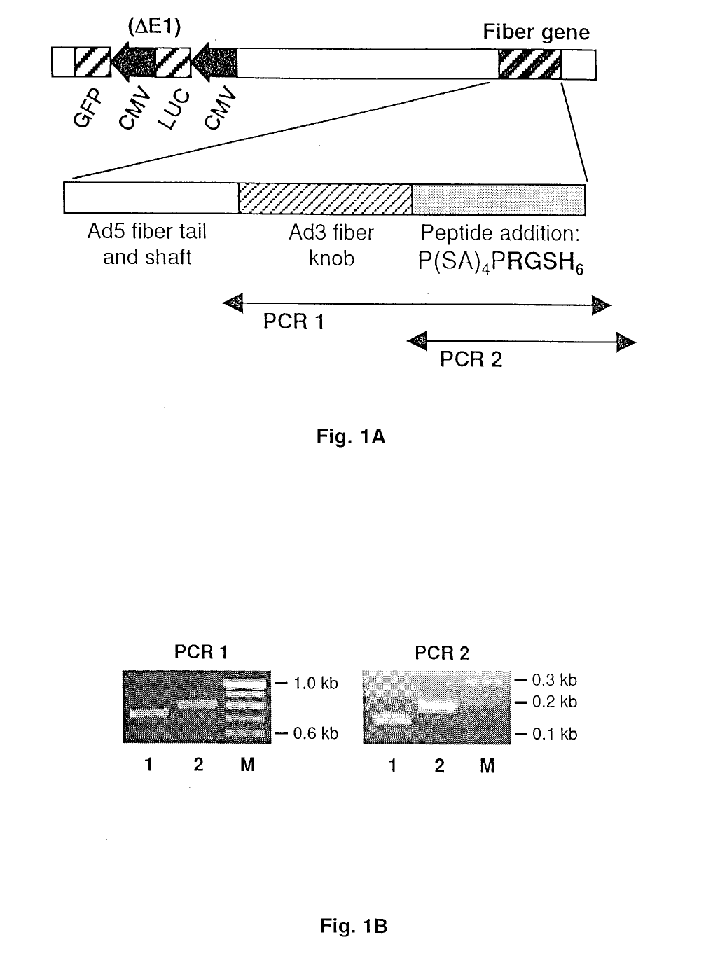

In the present example, two Ad5 based adenoviral vectors were modified by replacing the native fiber knob with an Ad3 fiber knob. These two vectors also contained within the E1 region an expression cassette consisting of a cytomegalovirus (CMV) promoter-driven green fluorescent protein (GFP) gene and a CMV promoter-driven firefly luciferase (LUC) gene (Ad5.F5/3 and Ad5.F5/3.Ct.His). Furthermore, in case of Ad5.F5/3.Ct.His, six His residues (preceded by a short spacer) had been genetically fused to the carboxy-terminus of the Ad3 fiber knob. Besides this ‘His-tag’ the two vectors were genetically the same (FIG. 1A).

These two modified vectors were constructed as follows: a plasmid containing the Ad5.F5/3 genome was generated by homologous DNA recombination between a PacI-KpnI fragment of pNEB.PK.F5/3 and a SwaI digested pVK50-8 based plasmid in E. coli BJ5183. pNEB.PK.F5/3 is a fiber shuttle vector containing a chimeric Ad5/Ad3 fiber gene (Krasnykh et al., 1996), whereas the pVK50-8 based plasmid contained the aforementioned GFP and LUC expression cassette in the E1 region (Seki et al., 2002). A plasmid containing the Ad5.F5/3.Ct.His genome was generated in a similar manner, except that pNEB.PK.F5/3 had to be first modified so that a short peptide linker—Pro-(Ser-Ala)₄-Pro and a six-His containing peptide Arg-Gly-Ser-His₆ would be added to the carboxy-terminus of the chimeric Ad5/Ad3 fiber. To this end a PCR technique was employed that in resulted in the introduction of the coding sequence 5′-CCATCAGCCTCCGCATCTGCTTCCGCCCCTAGAGGATCCCATCACCATCA C CATCAC-3′(SEQ ID No. 1) between the last coding codon of the chimeric Ad5/Ad3 fiber gene and its stop codon.

Adenovirus DNA was released from the generated adenovirus genome plasmids by PacI digestion and used for transfection of 293 cells to rescue the virus as described previously (Chartier et al., 1996). The viruses were rescued successfully, indicating that the heterologous addition to the Ad3 fiber knob was structurally compatible with correct folding and biological functions of the fiber molecule. The adenovirus vectors were propagated on 293 cells and purified by centrifugation in CsCl gradients by a standard protocol. Viral particle titers were determined spectrophotometrically by the method of Maizel et al. (1968), using a conversion factor of 1.1×10¹² viral particles per absorbance unit at 260 nm.

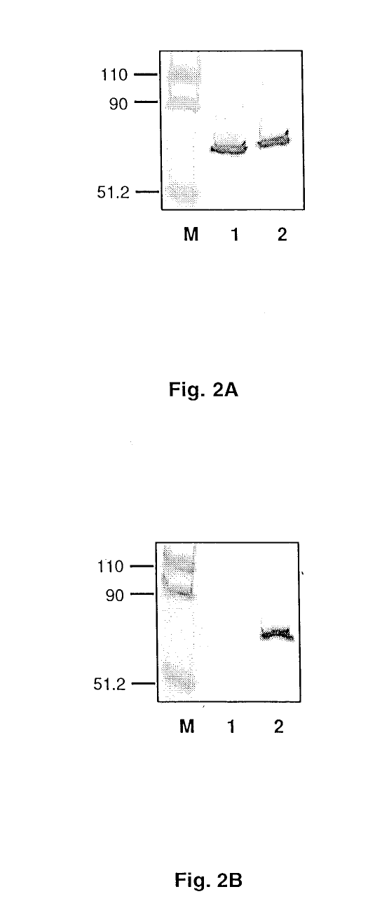

To verify the structural integrity of the fiber region of the viral genomes, DNA isolated from viral particles was analyzed by PCR. In both cases (Ad5.F5/3 and Ad5.F5/3.Ct.His) this resulted in the generation of amplification products of the expected lengths (FIG. 1B). Western blot (WB) analysis of denaturated viral particles demonstrated that the chimeric Ad5/Ad3 fibers had the predicted size (FIG. 2A). It was also verified that the carboxy-terminal His-tag was present on the fibers of Ad5.F5/3.Ct.His and absent on those of the control virus Ad5.F5/3 (FIG. 2B).

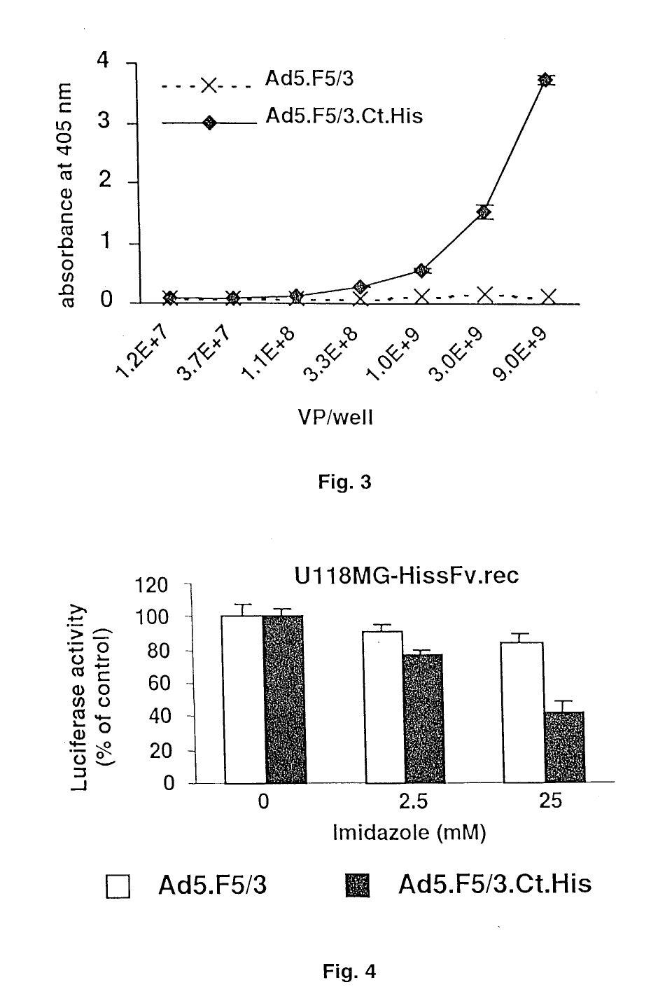

If the carboxy-terminus of the Ad3 fiber knob is to be used for re-targeting strategies, then it is of necessity that targeting moieties incorporated at this site are accessible for binding in the context of the intact virion. To investigate whether this was the case for the carboxy-terminal added His-tag, an enzyme-linked immunosorbent assay (ELISA) was performed. A range of three-fold dilutions of CsCl-purified virions (Ad5.F5/3 and Ad5.F5/3.Ct.His) immobilized in the wells of an ELISA plate were incubated with an anti-five-His mAb (Qiagen). Bound monoclonal antibody was detected by incubation with a goat anti-mouse IgG conjugated to alkaline phosphatase followed by development of the plate with p-nitrophenyl phosphate and reading at 405 nm. This analysis clearly showed efficient binding of anti-five-His antibody to immobilized particles of Ad5.F5/3.Ct.His, while binding to the control virus (Ad5.F5/3) was at the background level at every virus dilution (FIG. 3). These results demonstrate that the carboxy-terminal His-tags present on the Ad3 fiber knobs of intact virus particles were indeed accessible for binding and, therefore, potentially available for interaction with a cognate cell surface receptor.

Next it was determined whether the His tags on the Ad3 fiber knobs of Ad5.F5/3.Ct.His virions were capable of functioning as receptor-binding ligands and mediating gene transfer via a non-Ad3 receptor. This was addressed by Ad-mediated gene transfer assays (Seki et al., 2002) utilizing U118MG-HissFv.rec cells which exhibit surface expression of an artificial His-tag receptor (AR) with specificity for carboxy-terminal His-tags (Douglas et al., 1999; Lindner et al., 1997). Specifically, a blocking experiment was conducted that capitalized on the fact that the artificial receptor has affinity (K_{D}=4×10⁻⁴ M) for imidazole (Lindner et al., 1997). Results in FIG. 4 demonstrated that Ad5.F5/3.Ct.His gene transfer to U118MG-HissFv.rec cells was inhibited by imidazole in a dose-dependent manner, while this was not the case for Ad5.F5/3 gene transfer. This verifies that the modified virus, Ad5.F5/3.Ct.His, was indeed capable of infecting U118MG-HissFv.rec cells by means of a specific interaction between the carboxy-terminal His tag of the chimeric Ad5/Ad3 fiber protein and the artificial His-tag receptor.

In conclusion, the Ad3 fiber knob had not been previously explored for the presence of potential sites that can harbor heterologous targeting motifs. In the present example a heterologous ligand was added to the carboxy-terminus of the Ad3 fiber knob of an Ad vector. This genetic modification proved to have rendered the vector capable of mediating gene transfer via an alternate, non-Ad3 receptor. Thus, this work demonstrates that the carboxy-terminus of the Ad3 fiber knob is feasible as a locale for the introduction of novel tropism determinants.

### EXAMPLE 2

Knob Serotype Chimerism can Alter Ad Trophism and Enhance Ad Infectivity.

In order to test Ad vectors with knob serotype chimerism, two Ad5 vectors were designed in which the knob domain was replaced by the knobs of Ad serotypes Ad3 and Ad37 that are known to lack CAR-binding capability. It is hypothesized that routing the virus to the non-CAR Ad receptors of alternate Ad serotypes might allow tropism expansion with enhancement of infectious potency, especially in the context of target tissue relatively deficient in CAR.

The gene encoding the Ad5-37 chimeric protein was constructed essentially as described for Ad5/3 fiber gene (Krasnykh et al., (1996). The genes were transferred to the genomes of E 1 -deleted. firefly luciferase-expressing Ad5 by homologous DNA recombination in E. coli. The resultant genomes were then used to rescue the viruses of interest via transfection of 293 cells. The viruses generated, Ad5/3Luc1 and Ad5/37Luc1, were expanded on 293 cells, purified on CsCl gradient and utilized for a number of gene transfer analyses essentially as described before. AdSLuc1, a vector bearing the wild type fibers, was employed as a control.

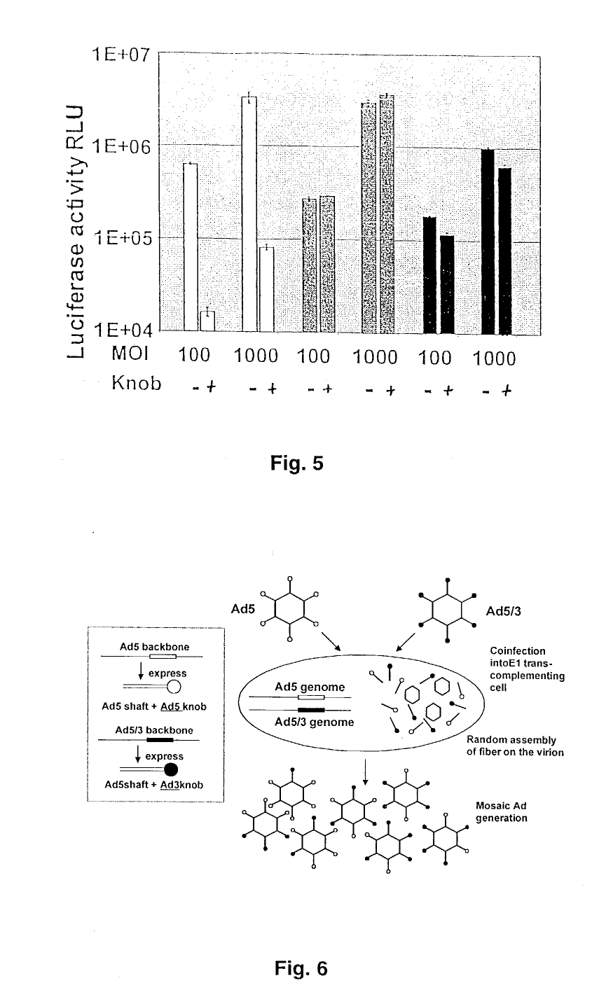

Gene transfer assays done on HUVEC cells showed that both Ad5/3Luc1 and Ad5/37Luc1 vectors containing chimeric fibers were able to achieve transgene delivery to this type of cell with efficiencies equal to that of the control virus incorporating wild type Ad5 fibers (FIG. 5). As expected, Ad5/3Luc1 as well as Ad5/37Luc1 binds to the cells in a largely CAR-independent fashion in that the binding was not blocked by recombinant Ad5 fiber knob (FIG. 5). These data suggest that replacement of the Ad5 knob domain with the knob domain of the Ad37 or the Ad3 fiber would result in expansion of the vector's tropism, which may be of utility for gene therapy applications.

### EXAMPLE 3

Mosaic Ad Vectors Expressing Both the Ad5 and Ad3 Knobs on the Same Virion

The present example describes a mosaic virus displaying both the Ad5 and Ad3 knobs on the same virion. The mosaic virus could utilize either the CAR and/or Ad3 receptor, resulting in enhanced infectivity. Compared to both the Ad5 and Ad5/3 vectors possessing knobs with a single receptor specificity, this mosaic Ad has significantly enhanced infectivity in various cancer cell lines and, thus, may offer the potential to improve Ad-based cancer gene therapy approaches.

The following abbreviations are used: CAR, coxsackie-adenovirus receptor; Ad5, serotype 5 adenovirus; Ad3, serotype 3 adenovirus; Ad5/3, Ad5 containing a chimeric fiber protein possessing the Ad3 knob; AdMS, mosaic adenovirus; Ad7, serotype 7 adenovirus; Ad5/7, Ad5 containing a chimeric fiber protein possessing the Ad7 knob; hCAR, human CAR.

### Materials and Methods

Cell Culture

Human cervical cancer cell lines HeLa and C33A, human non-small cell lung cancer cell lines NCI-H157 and NCI-H358, human pancreatic cancer cell line Panc-1, human breast cancer cell line AU-565 and the human glioma cell line U118 were obtained from the American Type Culture Collection (Manassas, Va.). The 293 human transformed embryonal kidney cell line was purchased from Microbix (Toronto, Ontario, Canada). Human ovarian adenocarcinoma cell line SKOV3.ip1 was obtained from Dr. Janet Price (M.D. Anderson Cancer Center, Houston, Tex.). Human ovarian adenocarcinoma cell line OV-4 was obtained from Dr. Timothy J. Eberlein (Harvard Medical School, Boston, Mass.). All cell lines were cultured in the recommended media at 37° C. in a humidified atmosphere of 5% CO₂.

Construction of Cell Lines Expressing Human Coxsackie-Adenovirus Receptor (hCAR) and an hCAR Truncation Mutant that Lacks the Cytoplasmic Domain

In order to assess the role of the cytoplasmic domain of CAR in infection by the mosaic Ad, a truncated form of human CAR (hCAR) lacking this domain was engineered. The 2.4 kb BamHI-NotI fragment carrying the hCAR cDNA was first subcloned from pcDNAI.hCAR (obtained from Robert W. Finberg, Harvard Medical School, Boston, Mass.) into the mammalian expression vector pcDNA3 (Invitrogen, Carlsbad, Calif.) A truncation mutant designated hCAR-tailless, in which a stop codon was inserted into the cDNA at the position corresponding to amino acid residue 261, was constructed by PCR mutagenesis using pcDNA3-hCAR as the template. The PCR product was inserted into the BamHI and NotI sites of pcDNA3 to give pcDNA3-hCARtailless. The integrity of the construct was verified by DNA sequencing.

Human U118 glioma cells which are refractory to Ad infection due to a paucity of CAR although they express the α_{V} integrins necessary for virus internalization were stably transfected with pcDNA3-hCARtailless or with pcDNA3-hCAR as a control. Individual single cell clones of U118-hCAR-tailless and U118-hCAR were isolated and expanded by selection in the presence of 400 μg/ml G418.

Recombinant Adenoviruses

Two replication-incompetent Ad vectors containing a firefly luciferase transgene cassette in place of the deleted E1 region were used. The Ad vectors Ad5luc1 and Ad5/3luc1 (Ad containing chimeric fibers with the tail and shaft domains of Ad serotype 5 and the knob domain of serotype 3) were constructed as described above and Krasnykh et al. (2001). The viruses were propagated on 293 cells and purified by two rounds of cesium chloride density centrifugation. The ratios of viral particle:infectious particles were 20 and 61 for Ad5luc1 and Ad5/3luc1 respectively.

Generation of Mosaic Adenovirus

Mosaic adenoviruses, designated AdMSluc1, were generated by co-infection of 293 cells with both Ad5luc1 and Ad5/3luc1. Monolayers of 293 cells in T75 flasks were coinfected with a total of 2×10⁹ particles of Ad5luc1 and Ad5/3luc1 in mixtures containing 100%, 90%, 70%, 50%, 30%, 10% or 0% of Ad5luc1. After infection for 3 hr at 37° C., the infection medium was replaced with fresh complete medium, and the 293 cells were incubated for 72 hr. The 293 cells were harvested and resuspended in 1 ml of DMEM:F12 medium containing 2% FBS and subjected to four freeze-thaw cycles. The crude viral lysates were used in the initial experiments. Based on the results of these preliminary experiments, a ratio of Ad5luc1:Ad5/3luc1 of 7:3 was chosen for coninfection of 293 cells for the large-scale preparation of the mosaic adenovirus AdMSluc1. Purification and titering were performed as described above.

PCR Amplification of Viral Genome

1×10⁸ viral particles of Ad5luc1, Ad5/3luc1 or AdMSluc1 were incubated in 0.1% SDS and 1 mM EDTA at 56° C. for 20 min to extract the viral DNA. The DNA was diluted 1/1000 in TE and 1 μl of this solution was used as a template for PCR. Each viral genome was amplified in a PCR reaction mixture (Qiagen, Valencia, Calif.) containing 50 nM primer pairs with 25 or 28 cycles of denaturation (94° C., 1 min), annealing (50° C., 1 min), and extension (72° C., 1min). The Ad5luc1 and Ad5/3luc1 backbone plasmids were used as positive control templates. The sequences of the primers were as follows:

Ad5knob-sense: 5′AGTGCTCATCTTATTATAAGA3′(SEQ ID NO. 2),

Ad3knob-sense: 5′CGCACATCCTATGTTATG3′(SEQ ID NO. 3),

knob-antisense : 5′CACCACCGCCCTATCCTGAT3′(SEQ ID NO. 4),

MLP-sense: 5′GGTTAATTAAGCATGTCCCTGACTCGCAT3′(SEQ ID NO. 5),

MLP-antisense: 5′TTGCGCGTGCACCTGGTGCCCGACGA3′(SEQ ID NO. 6).

To quantify the viral genome DNA in the crude virus solutions, the extracted DNA was amplified by real-time PCR as reported previously (Adachi et al., 2001). The viral DNA was isolated from 200 μl of each crude viral solution using a Blood DNA kit (Qiagen). Viral DNA was eluted with 100 ml of elution buffer [10 mM Tris Cl (pH 8.5)]. One microliter of eluted sample was analyzed by real-time PCR using a Light Cycler (Roche) to evaluate the Ad5 E4 copy number. Oligonucleotides corresponding to the sense strand of the Ad5 E4 region (5′-TGACACGCATACTCGGAGCTA-3′, 34885-34905nt, SEQ ID NO. 7), the antisense strand of the E4 region (5′-TTTGAGCAGCACCTTGCATT-3′, 34977-34958nt, SEQ ID NO. 8), and a TaqMan probe (5′-CGCCGCCCATGCAACAAGCTT-3′, 34930-34951 nt, SEQ ID NO. 9) were synthesized and used as primers and probe for real-time PCR analysis. The PCR conditions were as follows: 35 cycles of denaturation (94° C., 20 sec), annealing (55° C., 20 sec), and extension (72° C., 30 sec). An Ad backbone vector pTG3602 (Transgene, Strasbourg, France) was used to generate a standard curve for Ad E4 DNA copy number.

Western Blotting

Western blotting for fiber protein was performed as described previously (Pereboev et al., 2001; Seki et al., 2002). Briefly, 2×10⁸ purified Ad virions (Ad5luc1, Ad5/3luc1 or AdMSluc1) were diluted in 2×SDS sample buffer (Bio-Rad) and heated at 96° C. for 3 min. Virus samples were separated on a 4-to-12% gradient polyacrylamide gel followed by electrotransfer onto a polyvinylidene difluoride (PVDF) membrane. After blocking with TBST-casein (Tris-buffered saline [10 Mm Tris-HCl, pH7.4, 150 Mm NaCl] plus Tween 20 to 0.05% and casein to 0.5%), membranes were treated with mouse monoclonal antibody, mAb 4D2 (1:2000 dilution). The membrane was then incubated with goat anti-mouse IgG-HRP conjugate (DAKO, Carpinteria, Calif.). The color reaction was developed by incubation of the membrane with Sigma Fast diaminobenzidine (Sigma). Monoclonal Ab 4D2 directed against the tail domain of Ad5 fiber protein was generated at the University of Alabama at Birmingham Hybridoma Core Facility (Dmitriev et al., 2000; Karayan et al., 1994).

Recombinant Fiber Knob Protein Production

Recombinant Ad5 and Ad3 fiber knob proteins with N-terminal 6×His tags were expressed in E. coli using the pQE30 expression vector (Qiagen). To increase protein yields, the expressed Ad3 fiber knob protein was purified from inclusion bodies using the BugBuster Protein Extraction Reagent (Novagen, Madison, Wis.) inclusion body protocol as recommended by the manufacturer. The protein was further purified using the Talon metal affinity resin (Clonetech, Palo Alto, Calif.) as recommended by the manufacturer. The concentration of the purified proteins was determined by Bio-Rad DC protein assay (Bio-Rad, Hercules, Calif.). The ability of each knob protein to form a homotrimer was verified by Western blot of unboiled samples. The primary antibody used for detection was the monoclonal anti-polyhistidine clone HIS-1 antibody (Sigma) and the secondary antibody was peroxidase-conjugated goat antimouse IgG (DAKO).

Virus Binding to Immobilized Monoclonal Anti-AD5 Knob Antibody

Monoclonal antibody 1D6.14 which specifically recognizes the trimeric Ad5 fiber knob protein, has been described previously (Douglas et al., 1996). Monoclonal antibody (mAb) 1D6.14 or control mouse IgG (Sigma) were diluted in 50 mM NaHCO₃ (pH 9.6) at a concentration of 5 μg/ml and immobilized on a Nunc-Maxisorp ELISA plate overnight. After unbound mAb was removed, each well was washed three times with TBS-T (50 mM Tris-HCl, pH 7.6, in normal saline). Each well was then filled with 200 μl of DMEM: F12 media containing 10% FBS to block non-specific binding at room temperature for 2 hr. After blocking, each well was washed three times with TBS and used for virus binding. Viral particles of Ad5luc1, Ad5/3luc1, AdMSluc1, or a mixture of Ad5luc1+Ad5/3luc1 (8×10⁷ or 8×10⁸) in 80 μl of DMEM: F12 containing 2% FBS were placed in the well and incubated at room temperature for 2 hr. Then, 10 μl of the virus solution in each well was used to infect 1×10⁵ HeLa cells to examine the luciferase activity. The wells were then washed with TBS three times and viruses bound to the well were solubilized in 1×SDS buffer and applied to a gradient polyacrylamide gel. Western blotting was performed as described above.

Competitive Binding Assay

To investigate the ability of recombinant Ad5 and Ad3 knobs to block adenovirus infection, Ad5luc1 and Ad5/3luc1 were added to cells in the presence of purified knob proteins. Monolayers of C33A, SKOV3.ip1 and HeLa cells in 12-well plates were preincubated with increasing concentrations of Ad5 or Ad3 knob in 500 μl of DMEM: F12 containing 2% FBS for 10 min at room temperature. Ad5luc1, Ad5/3luc1 or AdMSluc1 were added at 100 or 1000 viral particles/cell in 100 μl of DMEM: F12 containing 2% FBS, and incubated for 1 hr at room temperature. The cells were washed once with DMEM: F12 containing 2% FBS, and maintained in complete media. After 48 hr of incubation at 37° C., the cells were lysed and luciferase assays performed as described below. In the experiment with the anti-AD5 knob monoclonal antibody, various amounts of 1D6.14 were preincubated with 1×10⁸ viral particles of each virus at room temperature for 30 min in a total volume of 20 μl HBS. Then, the virus-antibody complexes were diluted to 1 ml in DMEM: F12 containing 2% FBS, and 100 μl aliquots were added to 1×10⁵ cells in 12-well plates.

Ad-Mediated Gene Transfer Assays

One hundred thousand cells in 12-well plates were infected for 3 hrs at 37° C. at 10, 100 and 1000 viral particles/cell by adding Ad5luc1, Ad5/3luc1 or AdMSluc1 diluted in 500 μl of DMEM: F12 containing 2% FCS. Cells were washed once with DMEM: F12 containing 2% FCS, and incubated for 48 hr in complete medium. The luciferase activity in the cell lysate was determined with a luciferase assay kit (Luciferase Assay System; Promega) and a FB 12 luminometer (Zylux corporation). Background luciferase activities were subtracted from the readings.

Determination of Receptor Expression by Flow Cytometry

Cells grown in T75 flasks were washed with PBS, harvested by incubating with 0.53 mM EDTA in PBS, and resuspended in PBS containing 0.1% sodium azide and 1% BSA. Two hundred thousand cells were incubated with 1 ml of a 1:50 dilution of primary mAb RmcB for 1 h at 4° C. The mAb RmcB directed to human CAR was produced using a hybridoma purchased from ATCC (Fechner et al., 2000). An isotype-matched normal mouse IgG1 was used as negative control. The cells were washed once with 1 ml of PBS/BSA/azide and incubated with 1 ml of 1:100 dilution of the secondary FITC-labeled goat anti-mouse IgG (Jackson Immunoresearch Laboratories, West Grove, Pa.). Ten thousand cells were analyzed immediately by flow cytometry at the University of Alabama at Birmingham FACS Core Facility.

Generation of Mosaic Virus Possessing Ad5 and Ad3 Knobs

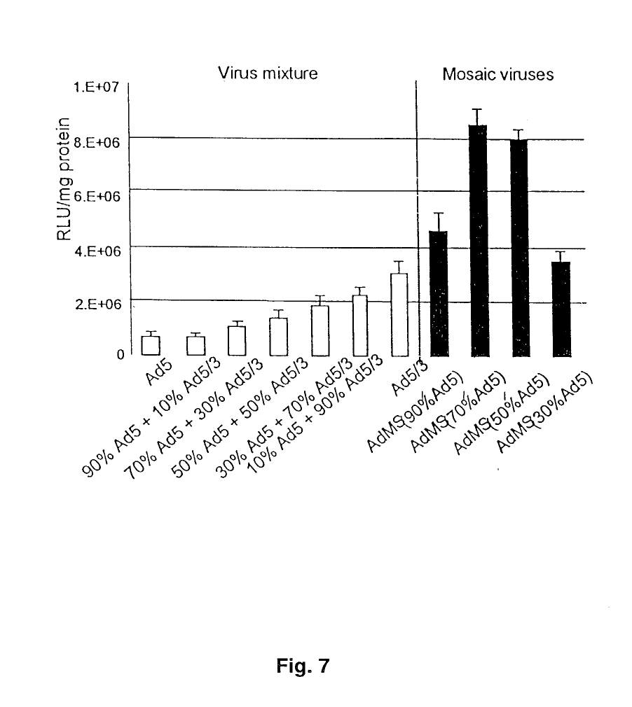

A set of four mosaic adenoviruses designed to display both the Ad5 and Ad3 fiber knobs was generated by co-infection of 293 cells with Ad5luc1 and Ad5/3luc1 at particle ratios of 90:10, 70:30, 50:50, 30:70 (FIG. 6). DNA was extracted from each crude virus solution and quantified by real-time quantitative PCR. An amount of each mosaic virus preparation, corresponding to 1×10⁸ copies of the E4 was used to infect 2×10⁵ HeLa cells. The parental vectors, Ad5luc1 and Ad5/3luc1, were employed as controls, together with various mixtures of these two vectors. The luciferase activities were measured after 48 hr as shown in FIG. 7. Ad5/3luc1 showed 4.2 times higher luciferase activity in HeLa cells than Ad5luc1, while the luciferase activities of the various mixtures of Ad5luc1+Ad5/3luc1 ranged between those of 100% Ad5luc1 and 100% Ad5/3luc1, increasing with the Ad5/3luc1 content of the mixture. Surprisingly, each of four mosaic viruses showed a higher luciferase activity than an equal number of particles of Ad5/3luc1 alone. The mosaic virus generated by coinfecting 293 cells at an Ad5:Ad5/3 ratio of 70:30 showed the highest luciferase activity in HeLa cells, 2.8 times and 12 times higher than Ad5/3luc1 and Ad5luc1, respectively. This observation encouraged us to perform quantitative analysis using the purified mosaic virus.

The Mosaic Virus Population Contains Two Separate Viral Genomes

Based on the preliminary results, the ratio of 70% Ad5luc1 and 30% Ad5/3luc1 was selected for large-scale production of the mosaic virus (AdMSluc1). The mosaic AdMSluc1, Ad5luc1, and Ad5/3luc1 were purified by two rounds of cesium chloride density centrifugation. The virus particle number (VP) of each purified virus was determined from the absorbance of 260 nm. PCR was performed with primers specific for either the Ad5 or Ad3 knob, while primers specific for the major late promoter (MLP) were used to quantify the viral genome. As shown in FIG. 8, the Ad5 knob-specific primers amplified only the Ad5luc1 and AdMSluc1 viral genomes, while the Ad3 knob-specific primers amplified only Ad5/3luc1 and AdMSluc1. The use of the MLP-specific primers indicated that equal amounts of viral DNA were employed as template. These results therefore confirm that the population of mosaic vectors, AdMSluc1, contained two separate viral genomes, derived from Ad5luc1 and Ad5/3luc1, which were encapsidated by the mosaic capsids.

Mosaic Virions Incorporate Both Ad5 and Ad5/3 Fibers

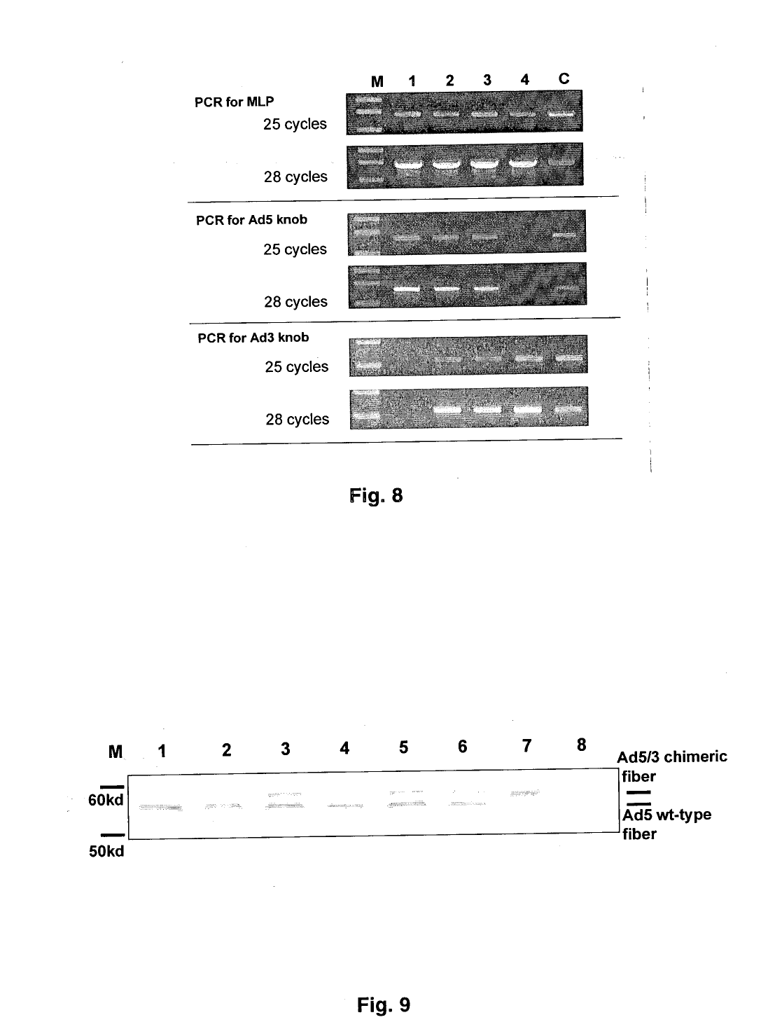

Since the two viral genomes incorporated into the mosaic virus population differ only in the knob region of the fiber, it was predicted that AdMSluc1 would contain two fiber proteins, the wild-type Ad5 fiber and the chimeric Ad5 fiber with the Ad3 knob domain, Ad5/3. To examine fiber incorporation during mosaic virus assembly, western blotting analysis was performed using monoclonal antibody (mAb) 4D2 that recognizes the common Ad5 shaft tail domain of both types of fiber. As shown in FIG. 9, this analysis of Ad5luc1 and Ad5/3luc1 revealed a single band corresponding to each fiber monomer protein (lanes 1 and 7). Fibers from Ad5luc1 and Ad5/3luc1 exhibited different electrophoretic mobility via SDS-PAGE and were identified easily by mAb 4D2. In contrast, AdMSluc1 and a 70:30 mixture of Ad5luc1+Ad5/3luc1 displayed two bands corresponding to the Ad5 wild-type fiber and the Ad5/3 chimeric fiber (lanes 3 and 5). In both cases, the intensity of the upper band which corresponded to the Ad5/3 chimeric fiber was weaker than the lower band that corresponded to the Ad5 wild-type fiber. Similar to the mixture of Ad5+Ad5/3, AdMSluc1 also seemed to have more Ad5 wild-type fiber than chimeric fiber. Following densitometric analysis of band intensity, lanes 1, 3, 5 and 7 were similar, indicating that equivalent numbers of viral particles contained a similar total amount of fiber protein.

These viruses were also applied to an ELISA plate for specific virus binding to an immobilized anti-Ad5 knob mAb 1D6.14. After washing, virus still bound to the wells was solubilized in SDS buffer and analyzed by SDS-PAGE. The lysate of Ad5luc1 bound to the immobilized mAb showed the same band as intact Ad5luc1 (FIG. 9, lanes 1 and 2). Lysate from the wells incubated with Ad5/3luc1 showed no band (lanes 7 and 8), indicating that mAb 1D6.14 is able to bind the Ad5 knob specifically without any cross reaction with the Ad5/3 chimeric fiber. Additional evidence was obtained about the specificity of mAb 1D6.14 from the lysate of the Ad5luc1+Ad5/3luc1 mixture. After the binding reaction with the mAb 1D6.14, only the Ad5 fiber protein was detected in the well incubated with Ad5luc1+Ad5/3luc1 mixture (lanes 3 and 4). Finally, the AdMSluc1 viruses were treated in the same way and both the wild-type and chimeric fiber proteins were detected in the mosaic virus lysates following binding to the ELISA plate (lanes 5 and 6). Based on these results, it was concluded that the majority of the virions in the AdMSluc1 population contain both the Ad5 wild-type fiber and the Ad5/3 chimeric fiber within the same particle.

Infectivity Profile of Mosaic Adenovirus in Various Cell Lines

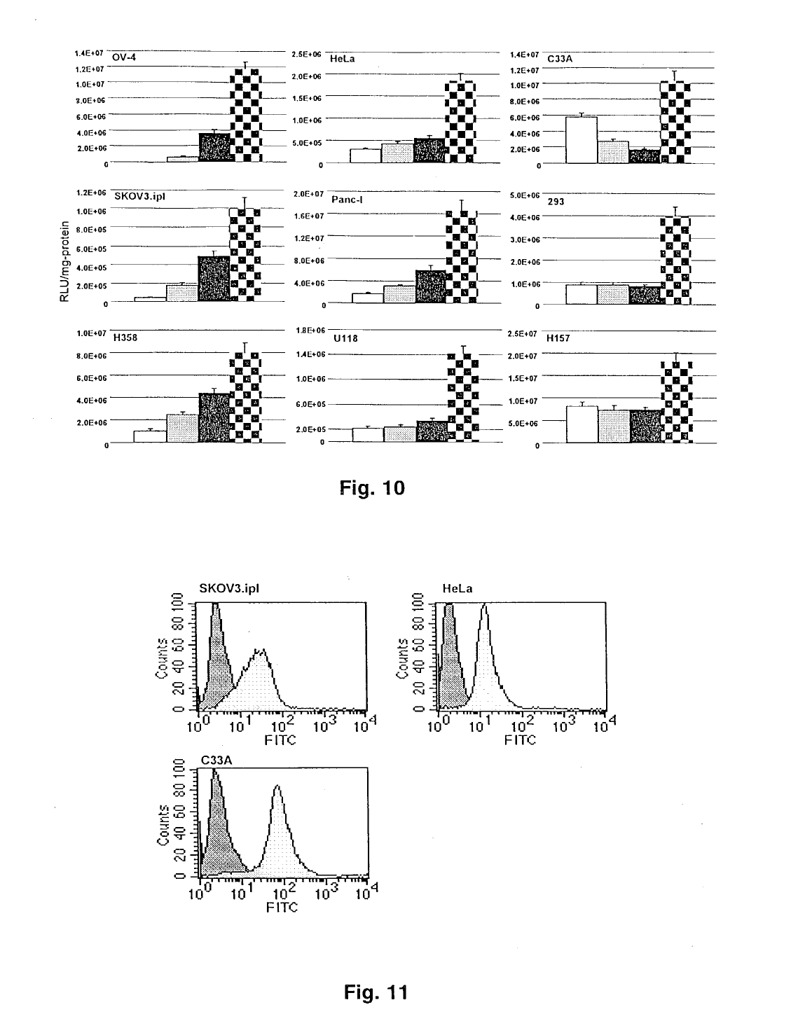

The mosaic virus demonstrated enhanced infectivity on HeLa cells compared with Ad5luc1 and Ad5/3luc1 (FIG. 7). However, it was unclear whether the infectivity enhancement was specific for this cell line. To address this question, the infectivity profile of AdMSluc1 in various cell lines was examined. Nine cell lines including ovarian cancer, non-small cell lung cancer, pancreatic cancer, glioma, cervical cancer and 293HEK cells were selected based on their expression of the primary receptors for Ad5 (CAR) and Ad3. After infection with each virus, the luciferase activities in this panel of cell lines were measured. It was reported previously that SKOV3.ip1 and OV-4 express the Ad3 receptor dominantly, while 293HEK cells express CAR as the dominant receptor. Consistent with these previous results, SKOV3.ip1 and OV-4 showed significantly higher luciferase activity with Ad5/3luc1 than Ad5luc1 (FIG. 10).

Conversely, some cell lines such as C33A, H157 and 293HEK cells were more susceptible to Ad5luc1 infection, presumably due to their dominant CAR expression.

The luciferase activity of the Ad5luc1 and Ad5/3luc1 mixture was also examined in each cell line and showed intermediate values between those for Ad5luc1 and Ad5/3luc1. No significant enhancement of infectivity was shown by this virus mixture. However, the luciferase activity with AdMSluc1 was superior to levels noted with Ad5luc1 and Ad5/3luc1 in all cell lines tested. AdMSluc1 showed a 300-fold increase in luciferase activity compared to Ad5luc1 in OV-4 cells. Likewise, AdMSluc1 showed 5.9 times higher luciferase activity than that of Ad5/3luc1 in C33A cells. These data revealed that the mosaic virus infected cells more efficiently than Ad5luc1 and Ad5/3luc1 regardless of the dominant knob receptor. Furthermore, the mosaic virus luciferase activity was higher than the sum total of luciferase activities with Ad5luc1 and Ad5/3luc1 in all cells tested, indicating that the presence of Ad5 and Ad3 knobs on the same virion creates a synergistic increase in Ad infectivity (the “mosaic effect”).

Analysis of Mosaic Ad Binding Using Purified Ad3 and Ad5 Knob Protein

In order to determine the manner in which the mosaic Ad utilized CAR and/or the Ad3 receptor during infection, infection blocking experiments were performed with purified Ad5 knob and/or Ad3 knob. C33A cells as a CAR-dominant cell line and SKOV3.ip1 as an Ad3 receptor-dominant cell line were used in these experiments. As indicated by FACS analysis as shown in FIG. 11, a higher percentage of C33A cells than SKOV3.ip1 cells are CAR-positive and a rough correlation was shown between CAR expression and luciferase activity with Ad5luc1 in these cell lines.

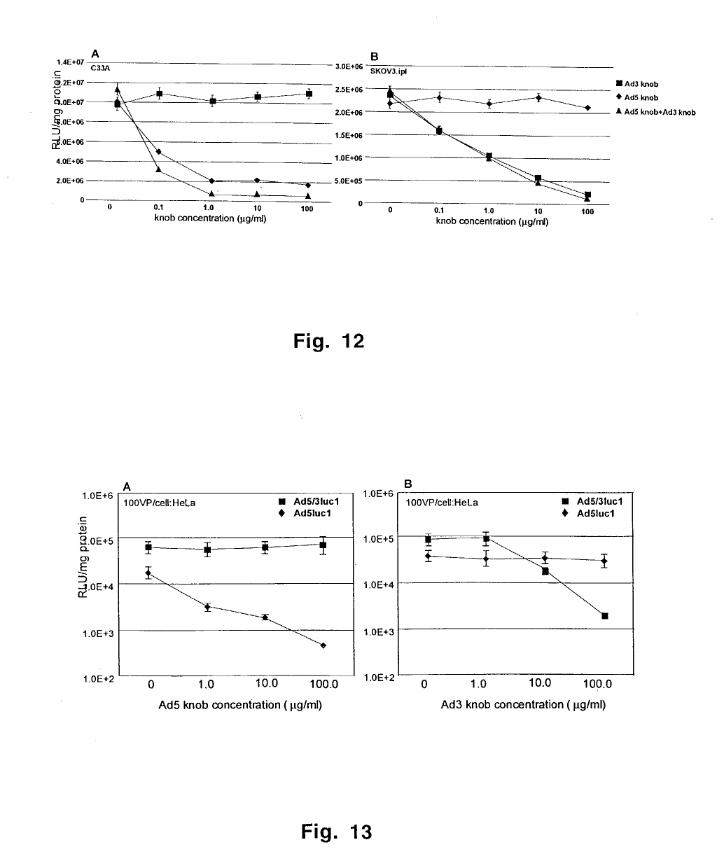

With C33A cells, purified Ad5 knob protein blocked gene transfer of mosaic Ad significantly at the lowest concentration of 0.1 μg/ml, and this blocking effect increased in dose-dependent manner as shown in FIG. 12A. Purified Ad3 knob protein did not show any suppressive effect even at the highest concentration of 100 μg/ml, but the combination of Ad5 and Ad3 knob protein blocked the mosaic virus infection more strongly than Ad5 knob alone. On the other hand, purified Ad3 knob blocked the mosaic virus infection in SKOV3.ip1 cells in a dose dependent manner as shown in FIG. 12B. Infectivity of the mosaic virus was not blocked by Ad5 knob in SKOV3.ip1 cells. Addition of Ad5 knob to Ad3 knob showed no additional effect in SKOV3.ip1 cells. These data demonstrated that the mosaic virus is able to bind to CAR-dominant cells with Ad5 wild-type fiber, and to Ad3 receptor-dominant cells with Ad5/3 chimeric fiber.

Virus Infectivity is Not Enhanced by Free Fiber Protein

Next it was determined if the engagement of one Ad receptor serotype by its cognate knob was sufficient to improve the infectivity of the virus bearing the other serotype knob. Therefore, infectivity enhancement of Ad5luc1 and Ad5/3luc1 in the presence of the respective free knob proteins was examined. HeLa cells were infected with Ad5luc1 or Ad5/3luc1 in the presence of various concentrations of Ad5 or Ad3 knob protein. As shown in FIG. 13, the luciferase activities with Ad5luc1 and Ad5/3luc1 infection were suppressed by recombinant Ad5 knob and Ad3 knob respectively in a dose-dependent manner. At a concentration of 100 μg/ml of each knob protein, the luciferase activities of the relevant adenovirus were below 5% of those without knob protein. Importantly, the luciferase activity of Ad5luc1 showed no change at the highest concentration of Ad3 knob protein. Likewise, Ad5/3luc1 infectivity was also not affected by Ad5 knob protein. Similar results were obtained when the same experiments were repeated with each virus at 1000 viral particles/cell (data not shown). Thus, these studies confirmed that each knob protein alone could not induce infectivity enhancement. Furthermore, this was also consistent with the previous result that the mixture of both viruses did not show any infectivity enhancement regardless of the mixture ratio as shown in FIG. 6.

Cytoplasmic Tail Domain of Coxsackie-Adenovirus Receptor (CAR) is Not Involved in the Mosaic Effect

The contribution of CAR-Ad3 receptor interaction to mosaic Ad infectivity enhancement was investigated. Any receptor interaction resulting in positive cooperativity of mosaic infectivity would require the intracellular domain of CAR. To directly address this issue, the CAR-negative U118 human glioma cell line and two genetic variants expressing either human CAR (U118-hCAR) or an hCAR deletion mutant lacking the intracellular domain (U118-hCAR-tailless) were utilized. Importantly, parental U118 cells and the two hCAR derivatives appeared to express the Ad3 receptor equally as evidenced by similar luciferase activities following Ad5/3luc1 infection (data not shown).

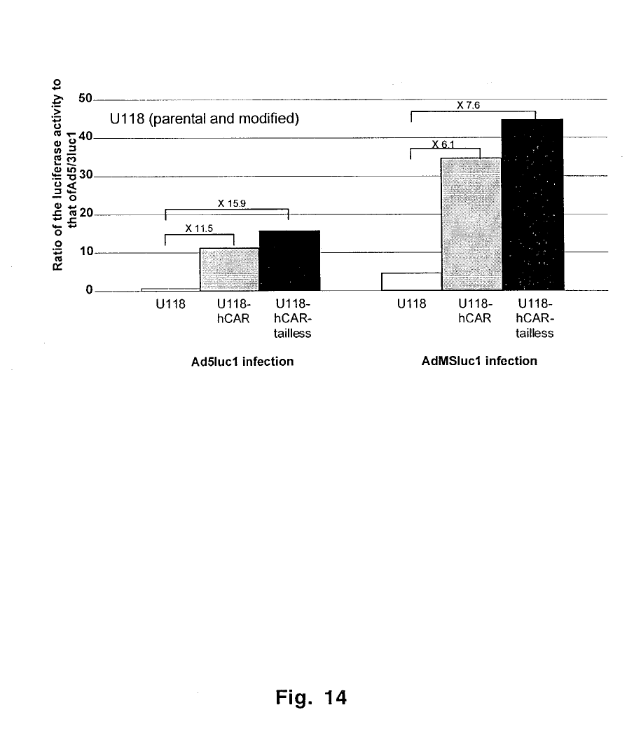

Initially, the luciferase activities of Ad5luc1 and Ad5MSluc1 in all three cell lines were compared as a percentage of Ad5/3luc1 luciferase activity for purposes of normalization (FIG. 14). As expected, Ad5luc1 transgene activity in the hCAR and hCAR-tailless expressing cell lines was increased 11-fold and 16-fold respectively compared to the CAR-negative parental line. Likewise, mosaic virus luciferase activity increased 6-fold in U118-hCAR cells and 7.5-fold in the U118-hCAR-tailless line versus parental U118 cells. Importantly, no reduction in AdMSluc1 infectivity in the U118-hCAR-tailless cells versus those expressing full length CAR was observed. These findings suggest that the CAR cytoplasmic domain does not contribute to the infectivity enhancement of mosaic virus. In other words, CAR and the Ad3 receptors do not interact with each other through their cytoplasmic tails to enhance the mosaic virus infectivity.

A major obstacle to be overcome in Ad5-based cancer gene therapy has been the paucity of the primary receptor, CAR, on human primary tumor cells. Variable expression of CAR has been documented in many cancer types including glioma, melanoma, bladder cancer, rhabdomyosarcoma, neuroblastoma and ovarian cancer. Moreover, it was also reported that CAR protein is down regulated in highly tumorigenic prostate cancer cell lines. Due to variable expression of CAR on human primary cancer cells, the utility of Ad5 as a cancer gene therapy vector is compromised, limiting overall efficiency of cancer gene therapy. On this basis, systems to circumvent intervening tumor-associated CAR deficiency are required.

In this regard, native Ad5 tropism can be modified to circumvent CAR deficiency and to enhance the adenovirus infectivity. One approach is pseudotyping, i.e., retargeting Ad by creating chimeric fibers possessing knob domains derived from alternate serotypes which bind to receptors other than CAR. To this end, nonreplicating Ads containing chimeric fibers with the tail and shaft domains of Ad serotype 5 and the knob domain of serotype 3 have been constructed (Krasnykh et al., 1996). It has been reported that a distinct Ad3 receptor existed on ovarian cancer cells based on a novel knob binding assay, and that the Ad5/3 chimeric vector was retargeted to the Ad3 receptor. Furthermore, competition assays with purified Ad5 and Ad3 knob protein suggested that the knob regions of Ad5 and Ad5/3 interacts with their cognate receptor independently. The present example describes a dual-knob mosaic virus possessing both Ad5 and Ad3 knob regions. In this regard, co-infection of 293 cells with Ad5 and Ad5/3 vectors gave rise to a population of viruses possessing both the Ad5 wild-type fiber and the Ad5/3 chimeric fiber on the same virion.

From competition assays with purified knob, it was confirmed that the mosaic virus can attach to either cellular receptor, CAR and Ad3 receptors. As expected, this mosaic virus enhanced Ad infectivity in various cell lines. The lucifease activity mediated by the mosaic virus exceeded the sum total of those with Ad5luc1 and Ad5/3luc1 in all cells tested, suggesting synergistic effect on infection. Interestingly, recombinant Ad5 knob protein did not block the mosaic virus infectivity in HeLa cells despite the fact that a significant suppression effect was observed with anti-Ad5 knob antibody. Taken together, the mosaic effect seemed to be synergistic rather than additive. Since the affinity of each knob for its cognate receptor is unchanged, the co-existence of both knobs on the same virion likely contribute to the synergistic infectivity enhancement.

A possible explanation for the synergistic infectivity enhancement is that binding of the Ad3 knob to the Ad3 receptor triggers an alteration in the CAR-Ad5 knob affinity, with the interaction between the two receptors inducing conformational or functional alterations of CAR. Previous studies with mutated CAR have reported that the cytoplamic tail functions as a sorting domain for basolateral targeting in epithelial cells, and it is not essential for adenoviral infection. However, other recent reports have suggested that overexpressed CAR acts as a tumor inhibitor in prostate cancer, and it might have an unknown function other than sorting. In this study, the comparison between the U118-hCAR cells and U118-hCAR-tailless cells indicated that CAR and the Ad3 receptor do not seem to interact with each other via the cytoplasmic domain.

Another possible explanation of the “mosaic effect” is a positional advantage of the Ad5 knob on the mosaic virus anchored to the Ad3 receptor. If the Ad3 receptor and CAR co-localize on the cell surface, the Ad5 knob may easily access to CAR close to the Ad3 receptor, allowing competition with recombinant Ad5 knob to be overcome.

The pathway of intracellular trafficking of Ad5 differs from that of Ad3 (Defer et al., 1990). Ad5 uses the cytoplasmic route preferentially after internalization, while Ad3 is sequestered within phagosomes. A study with an Ad5/7 chimeric vector possessing the Ad7 fiber on the Ad5 capsid reported that this Ad5/7 chimera traffics via the lysosomal pathway as well as that of Ad7. If the fiber protein modulates the virus trafficking pathway, the mosaic virus may utilize both pathways to the nucleus. This is another possible reason for synergistic enhancement of transgene expression. The precise mechanism for the mosaic effect observed in this study will be elucidated by the detailed analysis of Ad3 receptor once this has been cloned.

As a result of the presence of both the Ad5 and Ad3 knobs on the same virion, the mosaic virus displays expanded tropism covering the infectivity spectrum of both Ad5 and Ad3, and may be promising for cancer gene therapy approaches involving gene transfer to cells or tissues refractory to Ad5 and Ad3 infection. Transgene expression from various pre-existing Ad5-based vectors should be enhanced by this mosaic formation strategy in combination with Ad5/3-based vector. An Ad5/7 chimeric vector may also be available for mosaic virus formation since, like Ad3, Ad7 belongs to subgroup B. This mosaic virus strategy is an easy and feasible method for infectivity enhancement and tropism expansion.

Concerning the mosaic variation, the ratio of Ad5 and Ad3 knob number on the virion seems to be important as shown in FIG. 6. Since the mosaic virus population is heterogeneous, a part of the population may not contribute to the infectivity enhancement due to the deviated ratio of knob. This means that the data in this study may represent the total effect of a heterogeneous virus population with various knob ratios. Therefore, selection of the mosaic virus with optimal knob ratio could potentially improve the mosaic effect more intensely. If the spatial relationship between both knobs and their receptors is involved in the mosaic effect, alignment of each knob on the virion is responsible for it.

In conclusion, the present example describes a mosaic adenovirus generated by co-infection of 293 cells with Ad5 and Ad5/3 chimeric virus. This mosaic virus displays expanded tropism and enhanced Ad infectivity in various cancer cell lines. Thus, mosaic virus strategy has a possibility to improve the effect of cancer gene therapy in various contexts.

### EXAMPLE 4

Generation of Complex Mosaic Ad Vectors with Fiber Knob Chimerism Plus HI Loop Modifications

Serotype 5 Ad vectors which incorporate the knob domains of serotype 3 adenovirus as well as Ad vectors that incorporate the integrin-binding peptide RGD4C at the HI loop of the serotype 5 knob have been constructed. Modification of the fiber knob of serotype 5 were based on the crystal structure of the knob that was available at that time. Based on this information, a strategy of direct deletion/substation was employed for incorporation of the sequence encoding the peptide RGD4C within this locale. Whereas comparable structural information is not available with respect to alternative serotype fiber knobs, sequence homology suggests that a similar approach is rational.

Novel fiber genes can be derived employing methods reported previously. Briefly, fiber genes can be derived which are mosaic with respect to the knob (serotypes 3 or 37 substitute for type 5 knob) plus also contain sequences encoding the peptide RGD4C within the predicted HI loop site. Shuttle plasmids encoding these novel fiber genes can then be employed for derivation of replication-incompetent, E1A-deleted Ad vectors by methods described previously. Such vectors can encode various reporter genes that are used for subsequent analysis of gene transfer (e.g. E. coli β-galactosidase, firefly luciferase etc). These shuttle vectors are employed for homologous recombination with the corresponding packaging plasmid with subsequent derivation of the corresponding recombinant Ad vectors. Rescued plaques can be expanded and genomes of the resultant Ad vectors can be characterized for inclusion of the relevant complex mosaic fiber genes by restriction endonuclease digestion and sequence analysis.

Analysis of Gene Delivery Biology of Complex Mosaic Ad Vectors with Fiber Knob Chimerism Plus Loop Modifications

The rescued viruses can be analyzed for entry biology and gene delivery efficacy. Specific entry via a given Ad serotype receptor (e.g. 5, 3, or 37) may be ascertained by specific blockage with recombinant knob of the corresponding serotype. It is predicted that the HI loop containing RGD4C would allow gene delivery over-and above that mediated via the corresponding fiber knob. This “knob independent” gene delivery thus establishes the degree to which the distinct Ad modifications (serotype chimerism, HI loop integrin-binding ligands) can operate in a functionally integrated manner to achieve expanded tropism of the Ad vector. For these studies, the Ad vectors encoding the luciferase reporter and/or β-galactosidase reporter can be delivered to target cells. Studied vectors include control Ad5 (Ad5Luc1) and simple knob serotype chimeras (e.g. Ad5/3Luc1 and Ad5/37Luc1). In addition, variants of each of these vectors containing RGD4C at the HI loop can also be studied.

### EXAMPLE 5

Generation of Complex Mosaic Ad Vectors with Targeting Ligands at Multiple Capsid Locales

The strategy of fiber protein modification can be further complemented by modifications of the hexon protein. This represents a new strategy in Ad capsid alteration that has not been extensively developed so far. Compared to the fiber protein, a major advantage of the hexon protein as a ligand-presenting molecule is the abundance of the hexon molecules within the Ad5 virion. An Ad5 capsid incorporates just 36 fiber proteins, whereas there are 720 copies of the hexon in each Ad5 virion. Therefore, the hexon outnumbers the fiber protein by a factor of twenty, thereby strongly supporting the rationale of utilizing this component of an Ad virion for incorporation of targeting ligands.

The abundance of the hexons may prove to be especially important in the context of utilization of peptide ligands that are highly specific but do not always possess very high affinities for the relevant targets. This lack of high affinity of binding to the target cell may therefore be compensated by numerous copies of targeting peptides presented by the hexon protein. Most importantly, the modifications of the hexon are proposed as a strategy which does not replace, but rather complements the fiber modification-based approach. These two molecules can be designed independent from each other and may work in concert, resulting in synergistic effect of enhanced Ad infection.

Modifications of the hexon protein can be accomplished by genetic incorporation of DNA sequences coding for ligands into the hyper-variable regions (HVR) of the hexon gene. Recent crystallography studies on the structure of the Ad5 hexon protein have led to significant refinement of the three dimensional model of the hexon molecule proposed previously for the highly homologous Ad2 hexon protein (Rux and Burnett, 2000). The most important result of this recent work is the demonstration of the localization of the HVRs of the Ad5 hexon on the surface of Ad capsid. Hence, these HVRs possess two key features suggesting their utility for the purposes of the ligand presentation: (i) the structure of these regions of the molecule is highly variable, suggesting that variations in the structure are well tolerated by the molecule, and (ii) the HVRs are exposed to the surface of the virion, thereby facilitating the interaction between the ligand and a relevant target receptor.

The experimental schema used in the present example include: (i) generation of a series of shuttle vectors suitable for genetic modifications of the hexon protein HVR; (ii) employment of these newly derived shuttle plasmids for the derivation of recombinant Ad genomes by homologous recombination in E. coli, and (iii) rescue of the viral vectors of interest by transfecting 293 with the Ad genomes derived in (ii). In order to be able to easily manipulate the structure of any HVR of the hexon, a series of seven shuttle plasmids, each suitable for modification of one particular HVR, were constructed. All these plasmids can be derived from the previously made shuttle vector pNEB.HEX. Each of these vectors will contain an unique restriction site designed to simplify subsequent incorporation of the linker- and ligand-encoding sequences within the modified sequence of an HVR. A suitable restriction site is that of the type I restriction endonuclease BaeI which is unique in its capacity to excise its recognition sequence from the vector and simultaneously generate unique sticky ends suitable for subsequent cloning of either oligonucleotide duplexes or DNA fragments generated by “sticky-end PCR” technique. These new vectors can then be further modified to contain various linker sequences flanking the site of incorporation of the targeting peptides.

It was noted that sequences immediately adjacent to the targeting peptide incorporated within HVR5 of the hexon may have dramatic effects on the ability of the ligand to bind its cognate receptor (Vigne et al., 1999). With this in mind, several derivatives of the shuttle vectors described above can be constructed to incorporate linker sequences of different length and structure. For this, a portion of the Ad5 penton base sequence encoding an RGD-containing loop was chosen. As it has been shown by cryo-EM studies, the integrin-binding RGD tripeptide of the Ad penton base protein is localized at the apex of a long flexible loop. This structure facilitates the interaction between this peptide motif and an integrin molecule. Thus, segments of this loop can be examined in the context of the hexon HVR to identify optimal configurations of the insert that would be suitable for subsequent replacement of the RGD peptide with other peptide ligands. Specifically, “short” and “long” linker lengths will be evaluated.

This first analysis will determine the optimal site within the hexon for localization of an integrin-binding peptide for the achievement of CAR-independent gene delivery. This vector base will then be employed for the derivation of complex mosaic Ad vector which incorporate RGD4C at the HI loop of the fiber knob plus the indicated hexon locale. These vectors can be derived employing the fiber and hexon rescue systems described above (Table 1). These vectors will allow us to test the hypothesis that a ligand localized at multiple capsid locales may allow enhanced Ad infectivity via an optimized CAR-independent delivery capacity.

Analysis of Gene Delivery Biology of Complex Mosaic Ad Vectors with a Targeting Ligand at Multiple Capsid Locales

The rescued complex mosaic viruses (targeting peptide at HI loop plus optimal hexon site) can be analyzed with respect to entry biology and gene delivery efficacy. In the first instance, the degree of CAR-independence achieved via the incorporated peptide ligands can be determined by examining gene delivery to target cells in the presence or absence of recombinant serotype 5 knob, the vector base of the complex mosaic vector. Comparison between control vectors, vectors containing the integrin-binding motifs at the fiber HI loop locale, vectors containing the integrin-binding motifs at the hexon HVR locale, and vectors containing the motifs at both locales will provide an index of the degree of CAR independence achievable by each configuration of the motif.

The degree to which the configuration-linked CAR-independence can augment Ad infectivity for net gene transfer enhancement can be examined by using a panel of immortalized tumor cell lines expressing variable levels of coxsackie-adenovirus receptor. This analysis will provide a direct index of the functional infectivity enhancement achieved via the genetic capsid modifications. In view of the fact that the combination of distinct strategies of genetic capsid modifications has not been reported heretofore in the field, results from this analysis will provide information as to the degree to which these modifications may act additively or cooperatively towards the achievement of CAR-independent gene delivery.

TABLE 1
Complex Mosaic Ad Vectors

I. Knob Serotype Chimeras Plus Ligand at Fiber HI Loop

Ad5 RGD4C
Ad3/5 RGD4C
Ad37/5 RGD4C

II. Ligand at Hexon Locale

AdhexRGD4C HVR1, short linker or long linker
AdhexRGD4C HVR2, short linker or long linker
AdhexRGD4C HVR3, short linker or long linker
AdhexRGD4C HVR4, short linker or long linker
AdhexRGD4C HVR5, short linker or long linker

III. Ligand at Hexon Locale Plus Ligand at Fiber HI Loop

AdhexRGD4C HIRGD4C

Vectors listed under I will be evaluated for knob-dependent and CAR-independent gene transfer dynamics compared to relevant control. Vector listed under II will be evaluated for CAR-independent gene transfer to determine the optimal hexon site/configuration for ligand incorporation. Vector listed under III is constructed in the context of complex mosaic Ad with ligand at the hexon site and the fiber HI loop site.

### EXAMPLE 6

Application of Complex Mosaic Adenoviral Vectors in Squamous Cell Carcinoma of the Head and Neck

In spite of the availability of contemporary surgery and radiation therapy modalities, the overall survival among patients afflicted with advanced squamous cell carcinoma of the head and neck (SCCHN) has remained essentially static over the past three decades. This fact argues strongly for the development of novel therapeutic approaches for SCCHN.

In this regard, specific SCCHN disease features have suggested that it represents an appropriate target for the evaluation of gene-based therapeutics. Of special note, the surface accessibility of the tumor potentially allows for direct gene delivery for the implementation of a variety of cancer gene therapy approaches that are based on in vivo tumor transduction. For achievement of this gene delivery, recombinant adenoviral vectors have been employed based on their superior in vivo efficacy characteristics.

The object of the present example is to examine the augmented efficacy and specificity of the complex mosaic adenoviral vectors in the context of SCCHN. Appropriate murine models of SCCHN such as human tumors xenografts in SCID mice can be used to examine the therapeutic efficacy and toxicity of the complex mosaic adenoviral vectors. Therapeutic efficacy gained via the new vector configuration can be examined by a novel molecular chemotherapy approach consisting of the cytosine deaminase (CD)/5FC enzyme/prodrug system in combination with external beam radiotherapy (XRT).

Whereas altered adenoviral vector tropism is designed to increase the efficacy of tumor cell transduction, such modifications may likewise alter bio-distribution parameters with direct consequence in vector-associated toxicity. The pattern of ectopic vector localization of the tropism modified particles may be quite distinct from their unmodified counterparts. To address this issue, vector localization and trafficking in murine systems can be examined by PCR-based methods described below. It is anticipated that vector efficiency gained by the mosaic vectors described herein should allow the employment of lower vector dose that should decrease vector-associated toxicity based upon the intratumoral inoculum.

Several lines of evidence suggest that the immune response to adenoviral vectors is closely linked to the magnitudes of vector dose employed. It is thus anticipated that an additional benefit of the tropism modification strategies disclosed herein will accrue in terms of lower dose employed inducing diminished host anti-vector immunity. This is supported by the observation that some of the vector modifications have allowed effective tumor transduction in the presence of a pre-existent anti-adenoviral humoral immunity (Blackwell et al., 2000). Thus, this study would also examine certain immunologic aspects of the modified vectors disclosed herein. Taken together, these studies will provide key information with respect to the efficacy, toxicity and immunogenicity of the complex mosaic vectors.

Determination of Infectivity-Enhancement of Complex Mosaic Adenoviral Vectors in Murine Models of SCCHN

It is hypothesized that the vector modifications disclosed herein will allow enhanced tumor cell transduction in situ, resulting in enhanced therapeutic outcome in gene therapy approaches for SCCHN. To validate the tropism-modified adenoviral vectors can achieve enhanced in situ transduction of tumor cells, infectivity enhancements achievable in the context of in situ gene delivery mediated by the complex mosaic adenoviral vectors (CMAd) can be examined in comparison to unmodified Ad for SCCHN targets. For these studies, the human SCCHN tumor cell line SCC-4 can be established in SCID mice as xenografts. Results from the studies discussed above will establish the optimal complex mosaic adenoviral vectors for this cell line. When tumor nodules reach appropriate size, different experimental groups will receive CMAd or Ad vectors encoding luciferase or LacZ. These vectors can b e administered by direct, intratumor injection that parallels intended clinical use. Forty-eight hours post-injection, the animals will be sacrificed and the nodules harvested. For the study employing the luciferase reporter, the nodules will be homogenized and direct analysis of luciferase gene expression can be determined via luminometry, employing standard techniques.

Direct comparison of the magnitude of heterologous gene expression mediated by each vector configuration can be examined as follows. Tumor nodules are established by subcutaneous implantation of 1×10⁶ SCC-4 cells. Ten to fourteen days post-implantation, various doses (10⁵, 10⁶, 10⁷ particles) of the luciferase encoding control vector, AdCMVLuc, or its tropism-modified counterparts can be injected into the nodules in a volume of 5.0 ul. As comparable vector titers are employed for each vector modification, differential gene expression can be directly ascribed to enhanced infectivity deriving from a given vector modification.

The above analysis can be correlated with transduction frequency by direct study of the LacZ reporter. For these studies, CMAd or Ad encoding LacZ can be administered intratumorally as for the luciferase encoding vectors. Forty-eight hours post-delivery, the tumor nodules will be harvested and stained for the product of the LacZ gene via standard methods. This analysis will provide correlates between any augmented net gene transfer with the CMAd in the luciferase study with an actual increase in the number of transduced cells. The number of transduced cells can be determined by counting LacZ positive cells in X-gal stained tissue sections. This can be accomplished in a double-blinded manner whereby 100 cells are counted in at least 3 high-powered fields per group. Taken together, these studies will identify the precise vector configurations of CMAd that allow enhanced efficiency of gene delivery for SCCHN in the context of the clinically relevant in situ gene delivery schema.

Determination of Gene Transfer Efficacy of Complex Mosaic Adenoviral Vectors in Human Primary Tumor Material

Validation of the ability of these complex mosaic vectors to achieve enhanced infection of SCCHN target cells requires primary tumor cells as the necessary and sufficient analysis substrate because numerous studies have indicated that immortalized human cell lines are poor substrate for vector analysis. Direct validation of infectivity enhancement for primary human tumor material by the complex mosaic vectors will provide the rationale for their direct translation into human clinical trials.

Primary human SCCHN tumor samples obtained during surgery at the University Hospital of the University of Alabama at Birmingham will be transported to the laboratory and processed for experimentation. Tumor and normal tissue are finely minced, distributed into approximately equal aliquots, weighed, and then overlaid with 100 μl of OptiMem (Gibco BRL, Life Technologies Inc). Normal tissues are defined as tissues that appear clinically normal and taken from the mucosa or underlying submucosa at a site that is at least 2 cm from the resection margin. For all experiments, 10 to 50 mg of tissue are used. For some experiments, a cell suspension is made by incubating the minced tumor tissue in Dispase protease solution (Worthington Biochemical Corp, Lakewood, N.J.) and then filtering through glass wool. The cells are then grown in cell culture medium at 37° C. Twenty-four hours later, the cells are subjected to Ad injection.

Determination of Therapeutic Utility of Complex Mosaic Adenoviral Vector in Murine Models of SCCHN

The therapeutic utility of infectivity enhancement mediated by the complex mosaic adenoviral vector can be studied as follows. For this analysis, a gene therapy approach based on molecular chemotherapy in combination with external beam radiotherapy will be used. This consists of intratumoral delivery of the cytosine deaminase gene (CD) in conjunction with the 5-FC prodrug followed by external beam radiotherapy (XRT). The therapeutic efficacy of this approach has been shown in murine models of loco-regional carcinoma.

Thus, after identification of the optimal retargeting schema for SCCHN tumor cells in vivo as described above, a version of this vector encoding the CD gene can be constructed. The recombinant Ad vector can be confirmed via restriction analysis and PCR. In addition, direct confirmation of CD expression can be obtained via in vitro tumor cell transduction followed by Western blot analysis for the product of the CD gene.

After validation, an unmodified Ad vector encoding the CD gene, AdCD, can be compared to a tropism-modified counterpart, AdCMCD. Xenografts of the human SCCHN tumor cell line SCC-4 can be established in SCID mice as described above. When the tumor nodules reach appropriate size, various doses (10⁵, 10⁶, 10⁷ particles) of AdCD or AdCMCD vectors are administered by direct intratumoral delivery. These two groups are then subjected to relevant treatment regimen consisting of 5FC followed by a course of external beam radiotherapy according to published methods. Some animals are treated with the viruses one time only, followed by standard 5FC and external beam radiotherapy. Alternatively, the animals will receive the indicated dose of virus daily, for five injections. Direct analysis of these study groups will allow determination of the growth dynamics of the treated tumor nodules. Enhanced tumor cell transduction achieved via the complex mosaic adenoviral vector allows enhanced CD expression and thus an improved therapeutic outcome.

Subsets of the experimental groups are subjected to analysis of intratumor CD expression via Western blot analysis of total tumor cell lysates. An important aspect of the present invention is that infectivity-enhancement achieved by the complex mosaic adenoviral vector will ultimately allow employment of lower vector doses to achieve a therapeutic effect. Thus, the above experiments can be repeated at incrementally lower viral doses of AdCD versus AdCMCD to examine the therapeutic effects.

In vivo Localization of Complex Mosaic Adenoviral Vectors in Murine Model of SCCHN

Gene therapy for SCCHN via adenoviral vectors is accomplished via in situ gene delivery to a localized tumor. In this delivery schema, tumor targeting is assumed to occur largely on the basis of local vector delivery. Nonetheless, human trials have demonstrated that even in this delivery schema, ectopic vector localization may occur. As the present invention is modifying aspects of the viral vector's cellular entry mechanism, it is also likely that other aspects of its in vivo distribution may differ from the unmodified adenovirus. Thus, in vivo distribution of the complex mosaic adenoviral vector (CMAd) in comparison to the unmodified Ad subsequent to intratumoral delivery can be examined as follows.

Viral vectors distribution can be monitored by a PCR-based methods employing the highly sensitive and reproducible quantitative techniques achieved via the TaqMan apparatus. Initially, subcutaneous xenografts of SCC-4 cells are established in SCID mice. After appropriate engraftment, intratumoral injections of Ad and CMAd are accomplished in a manner that mimics clinical treatment protocol. At distinct intervals post-injection, the animals are sacrificed, major organs harvested, and the levels of the vector genome are determined at each organ site by quantitative PCR employing the TaqMan apparatus. Studies are carried out to evaluate localization at early time points post-treatment (48 hours) and at late periods (2 weeks).

In addition to the intratumoral injection schema, the fate of the complex mosaic adenoviral vectors can also be examined in the context of intravenous delivery such as tail vein injection. Although this delivery schema does not precisely parallel planned human use, this information may be important in establishing the fate of the viral particles that escape the local tumor inoculum. Taken together, these studies will establish the in vivo distribution which occurs as a result of the tropism modification described herein. Such information may be of key relevance in conceptualizing the types of anti-cancer genes that may be employable for SCCHN gene therapy, and in understanding their toxicities.

Toxicologic Analysis of Complex Mosaic Adenoviral Vectors in Murine Models of SCCHN

The potentially altered biodistribution of the complex mosaic adenoviral vectors may also effect their toxicologic profile compared to the unmodified Ad vectors. In this regard, it is critical to establish that maneuvers to enhance Ad infectivity for tumors have not also increased the vector's toxicity. To this end, basic toxicology studies can be performed to exclude the possibility that the therapeutic index may be negatively impacted by the tropism modification maneuvers.

Initially, a murine model of SCCHN (human xenograft in SCID mouse) is treated with intratumoral injections of Ad and complex mosaic adenoviral vector at doses of 10⁵, 10⁶, 10⁷, 10⁸, and 10⁹. Treated animals are evaluated for any acute clinical changes that suggest toxicity. Specifically, a formal LD₅₀ for the complex mosaic adenoviral vector (CMAd) will be defined for comparison to unmodified Ad. In addition, animals will be sacrificed forty-eight hours post-treatment and histopathologic analyses of major organs can be performed to determine the presence of any end organ changes indicative of inflammation/necrosis. These studies will determine any gross toxicity differences between Ad and CMAd in the context of a treatment schema mimicking the planned human context.

Similar database can also be established in immunocompetent mice. In this regard, the relevant FDA database for toxicologic analysis of Ad vectors is in the context of C57BL/6 mice. Thus, standard FDA analyses of complex mosaic adenoviral vector versus Ad can be performed in immunocompetent mice by administering various doses of virus (e.g. 10⁷, 10⁸, 10⁹, 10¹⁰) via various routes (e.g. intravenous, subcutaneous, intraperitoneal), and studying clinical parameters, hematologic/serologic parameters, and histopathologic parameters. These studies will thus establish any potential differential toxicities associated with the SCCHN targeted Ad. Furthermore, this database will establish an important point-of-departure for evaluation of an ultimate therapeutic agent for SCCHN clinical trial which embodies complex mosaic adenoviral vector components.

Immunologic Analysis of Complex Mosaic Vectors

SCCHN gene therapy involves intratumoral gene delivery via adenoviral vectors. Of note in this regard, several groups have demonstrated that intratumoral adenoviral vector-mediated gene delivery could be effectively achieved even in the context of a pre-existent anti-Ad humoral immunity. This fact has been noted in the context of syngeneic murine models of carcinoma as well as in human trials.

One of the potential benefits derived from targeting strategies is the mitigation of anti-vector immunity. This is based on the concept that priming of the immune system against the vector is facilitated by vector uptake by dendritic cells (DCs). Thus, the degree to which a vector may be “un-targeted” to DCs may be an important parameter predicating its reduced immunogenicity. A preliminary examination of the immunologic consequences of the tropism modification strategies disclosed herein can be obtained by experiments in immunocompetent murine systems. Initially, the magnitude of induction of humoral and cell-mediated immune response to complex mosaic adenoviral vector (CMAd) versus unmodified Ad can be examined by inoculating C57BL/6 mice with either CMAd or Ad. In addition, similar studies can be carried out in animals that have been pre-immunized with CMAd or Ad. These studies will provide direct qualitative and quantitative indices of any differential immune response elicited to CMAd versus Ad. In addition, these studies may serve as the foundation for establishing the pre-clinical rationale for evaluation of the CMAd in a human clinical context.

### EXAMPLE 7

Application of Complex Mosaic Adenoviral Vectors in Pancreatic Cancer

In spite of the availability of contemporary surgery and radiation therapy modalities, the overall survival among pancreatic cancer patients has remained essentially static over the past three decades. This fact argues strongly for the development of novel therapeutic approaches for pancreatic cancer.

Gene therapy for pancreatic cancer represents a means to potentially achieve effective management of loco-regional recurrences/complications, and surgical accessibility of pancreatic tumor potentially allows for direct gene delivery for in vivo tumor transduction. Based on their superior in vivo gene delivery characteristics, recombinant adenoviral vectors have been employed to deliver therapeutic genes to pancreatic cancer cells. These approaches have included delivery of the p53 tumor suppressor gene or toxin encoding genes such as HSV-Tk and cytosine deaminase. Although adenoviral vectors are understood to exhibit superior levels of in vivo gene transfer compared to alternative available vector systems, their present level of efficiency nonetheless is suboptimal for pancreatic cancer gene therapy applications due to deficiency of the primary adenovirus receptor, CAR, on the tumor cells.

The present invention discloses a novel approach to enhance adenoviral gene delivery via simultaneous capsid incorporation of multiple and distinct retargeting ligands. It is hypothesized that such complex mosaic vectors would improve gene delivery to pancreatic cancer cells and improve therapeutic index for pancreatic cancer gene therapy. The efficacy and specificity of the complex mosaic adenoviral vectors in the context of pancreatic cancer can be examined using the methodologies described above in the context of SCCHN. Appropriate murine models of pancreatic cancer such as human tumors xenografts in SCID mice can be used to examine the therapeutic efficacy and toxicity of the complex mosaic adenoviral vectors. In vivo localization and immunologic analysis of complex mosaic adenoviral vectors can also be examined in murine models of pancreatic cancer using the methodologies described above.

### EXAMPLE 8

Adenoviral Vectors with Targeting Ligand At the Fiber HI Loop Achieve Enhanced Gene Delivery to Cardiovascular Targets

An adenoviral vector with targeting ligand at the fiber HI loop was constructed. This vector, Ad5lucF_{HI}RGD, contained fibers incorporating an RGD-4C peptide within the HI loop of the knob domain. Insertion of this peptide has been shown to allow the vector to achieve CAR-independent attachment to target cells via specific interaction of the RGD peptide with the cellular integrins.

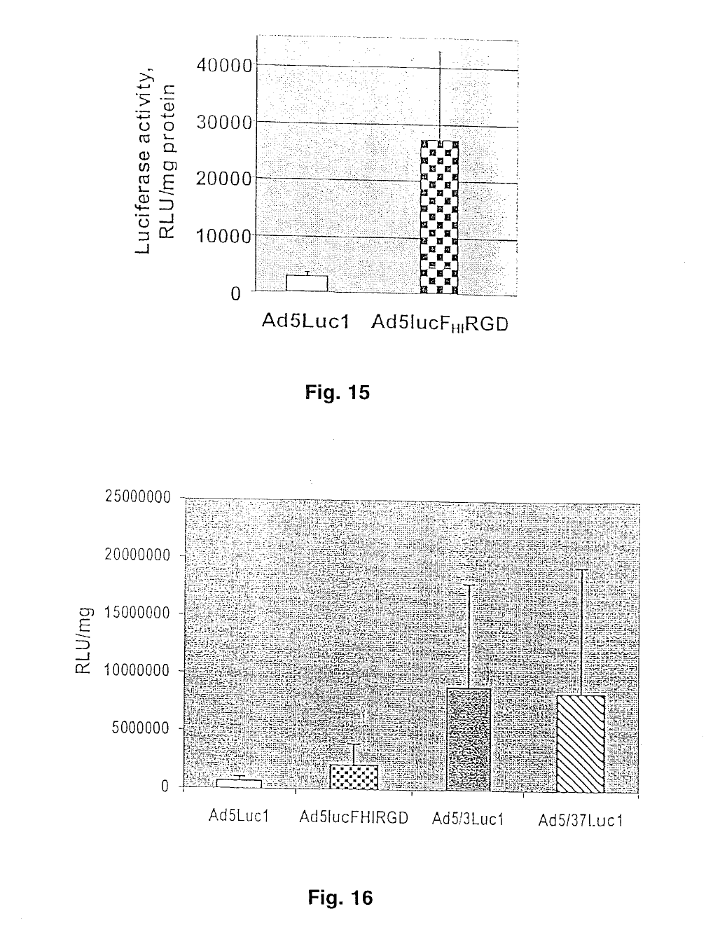

Ad-mediated gene transfer to human heart tissue samples was done as follows. After removal from the patient, the heart was immediately placed in the preservative (University of Wisconsin Solution) on ice and transported to the laboratory. Cardiac muscle was dissected into small pieces of approximately 1 mm³ each that was plated at four pieces per well into 24 well plates. Six replicate wells were then infected with either Ad5Luc1or Ad5lucF_{HI}RGD for 30 at 10⁹ viral particles per ml. The tissues were then washed and incubated in complete media at 37° C. for 24 hours to allow for reporter gene expression. The tissues were then lysed in a lysis buffer (Promega) compatible with the luciferase assay. Results shown in FIG. 15 clearly shows that the modified Ad vector was several fold more efficient in infecting heart tissue than unmodified vector. In addition, vectors with knob chimerism were also analyzed for gene delivery to human heart. As shown in FIG. 16, it is apparent that Ad vectors with Ad3 or Ad37 knob chimerism accomplished enhanced gene delivery to human heart tissues.

### EXAMPLE 9

Application of Complex Mosaic Adenoviral Vectors in Cardiovascular Diseases

The definition of the pathobiology of a variety of cardiovascular diseases has allowed the recent application of molecular therapeutics for treatment of a subset of these disorders. In this regard, an exciting new option, “therapeutic angiogenesis”, based on the induction of new blood vessel growth into poorly perfused, ischemic myocardium has recently been developed (Simons et al., 2000). In essence, this approach is based upon knowledge of the signals involved in the biology of vessel formation and appropriate angiogenic factors are delivered to cardiac muscle. Several angiogenic factors such as vascular endothelial growth factor (VEGF) and fibroblast growth factor (FGF) have been studied in animal models. These studies have established the feasibility of the approach for atherosclerotic coronary artery disease (CAD); however, a key issue in the practical application of this therapy for human use is the mode of delivery of the angiogenic agent. An attractive means to achieve local and continuous release of these agents is to deliver via adenoviral vectors the genes for the relevant factors rather than the proteins themselves.

Despite the utility of adenoviral vectors for this clinical application, a number of vector-related issues have become apparent in human clinical trials that may limit the realization of the full value of this genetic intervention for coronary artery diseases. In this regard, ectopic localization of Ad vector expression may be associated with expression of the therapeutic transgene at non-target sites. Furthermore, a clear dose-related toxicities have been noted with Ad vector employment. Concerns about adenoviral toxicity were dramatically heightened in late 1999 when the first death of a patient in a gene therapy trial occurred as a direct result of the administration of a large dose of adenoviral vector. It is important to note that the whole gene therapy scientific community has undergone a careful reconsideration of gene delivery issues following this event. The positive and negative attributes of adenoviral vectors in particular have been carefully weighed and a consensus has emerged. Currently, these agents remain the most efficacious gene delivery agents for in vivo uses, and as such, further research and development of these vectors are not only warranted but possibly key to the realization of many gene therapy approaches.

In view of the established association between dose and toxicity, it is clearly rational that adenoviral vectors that are intrinsically more efficient than the basic adenovirus could be used at much lower doses, thereby reducing the toxic effects. Moreover, the combination of lower initial dose plus greater fractional proportion of the dose being retained at the administration site will naturally result in much less risk of dissemination, either locally through the myocardium or systemically. Such infectivity-enhanced vectors would therefore also allow for much more precise localization of angiogenic effects that can be coupled with improvements in expression cassette design to address concerns of poorly controlled angiogenesis and potential problems that arise from that.

With these consideration in mind, the present example present genetic capsid modifications of adenoviral vectors to achieve optimized gene delivery to myocardial target cells. These modified vectors infect target cells via cellular markers over-and-above the native Ad receptor coxsackie-adenovirus receptor (CAR). Such CAR-independent gene delivery results in dramatic enhancement of Ad vector infectivity that would allow employment of lower Ad doses for the achievement of genetic intervention. By addressing a key issue that limits the translation of present gene therapy strategies into clinical trials, the development of such a vector system therefore represent a major technological advance in gene therapy for cardiovascular disease.

Analysis of Gene Delivery to Primary Human Heart Material Mediated by Complex Mosaic Ad Vectors

As it has been discussed above, a variety of considerations predicate the utility of primary human tissues as the necessary and sufficient analysis substrate for the following experiments. A system has been established at University of Alabama at Birmingham whereby human hearts removed from transplant recipients will be collected immediately upon removal from the patient. Following removal from the patient, the entire heart (excluding material required for pathological analysis as part of the primary care of the patient) is placed in the preservative University of Wisconsin (UW) solution at 4° C. and transported immediately to the laboratory. The heart will be quickly dissected (while maintained at 4° C. in UW solution to preserve viability) into various anatomic regions corresponding to known previous diseases.

For immediate gene transfer assays, samples of heart muscle obtained from the various regions will be finely minced, placed into 24 well tissue culture plates and incubated in specialized medium containing relevant factors that enhance viability. Spontaneous contractions of myocytes can be observed as an index of their viability. Cells in replicate wells (4-6) are infected immediately using candidate and control vectors, each of which will be used in at least three serial dilutions. The principal reporter system used initially for each vector will be luciferase, based on the high sensitivity and quantitative nature of the enzymatic assay. Furthermore, replicate wells will also be assayed using an anti-luciferase antibody and immunohistochemistry to identify the cell types being infected.

The gene delivery biology of the complex mosaic Ad vectors in the context of human heart target cells can be examined as follows. Specific blockade reagents such as recombinant knobs of human Ad serotypes 3, 5 and 37 will be used to characterize the contribution of various cellular pathways to the infection biology of the complex mosaic Ad vectors. Fresh primary human myocytes as described above are used for infection studies in the presence or absence of specific blockade. These studies will establish the biologic basis of relative vector efficacy and provide pivotal information relevant to the choice of vector species for human clinical translation.

To Evaluate the Safety and Toxicity Issues of Complex Mosaic Adenoviral Agents in the Context of Human Heart Target Cells

One of the basic premises of the approach disclosed herein is to reduce vector-related toxicity by reducing the absolute amount of vector required to achieve a therapeutic impact. Vector related toxicities arise in several ways, incorporating innate immune mechanisms, antibody and cell-mediated immunity. It is plausible that differential infection profile the tropism-modified vectors will achieve could potentially have unforeseen toxicities. These toxicities could arise either from responses to the Ad vector per se or from the differential distribution of the angiogenic transgenes delivered by the vectors. Thus, preliminary toxicity studies can be performed as described below in well-established murine models as a necessary first step in screening for such unforeseen eventualities. These studies will evaluate tropism-modified vectors containing reporter genes and those containing genes for angiogenic factors.

Evaluation of Vector Biodistribution Following Systemic Vascular Administration in Mice

C57BL/6 mice (n=5 per group) will be injected via the tail vein (doses of 10⁷, 10⁸, 10⁹, 10¹⁰ or 10¹¹ viral particles) with infectivity-enhanced vectors or native control vectors each carrying the gene for the luciferase reporter. Ninety minutes later the mice are sacrificed and various organs such as lung, heart, liver, spleen, kidney, ovary (or testis), small and large intestine, thyroid, brain, muscle and peritoneum will be harvested and snap-frozen. DNA can be extracted from these tissues using standard techniques. Vector-specific DNAs are quantified using TaqMan PCR and referenced to a standard curve. Some of these animals will be sacrificed three days later, then various organs can be harvested and assayed to determine the luciferase activity. These studies will ascertain whether any unexpected biodistribution has taken place, and provide important information concerning both vector particle distribution and the transgene expression profile of the new vectors. These data will also serve to focus subsequent toxicity analysis on a particular organ or system.

Biochemical and Histological Analysis of Toxicity Following Systemic Administration.

In these studies, mice are injected as described above with vectors containing the luciferase reporter gene or genes for angiogenic factors. The animals will be sacrificed at 3, 5, 7, 10 or 14 days post-injection. Various organs are harvested as above, fixed in formalin and processed for standard H & E staining. At the time of sacrifice, blood samples are also collected for full biochemical analysis including electrolytes, liver enzymes (AST, ALT, GGT, LDH, bilirubin), urea, creatinine, and muscle enzymes, CK. These studies will identify any particular organ toxicities both due to the vectors per se and as a response to the delivered angiogenic factor.

Assessment of Toxicity Following Local Intramuscular Administradon

The eventual application of the vectors clinically will be in a local administration context applied directly to the myocardium. Thus, preliminary toxicity studies will be performed in a local delivery context by administering the vectors intramuscularly to mice, then assessing for local inflammatory reactions and the occurrence of necrosis. As before, C57BL/6 mice are used for this study. These animals (n=5 per group) will receive injections of tropism-modified or control vector in doses ranging from 10⁷ to 1o¹¹ particles by direct injection into the thigh muscle. Groups of animals are sacrificed at various time points as above, then muscle from the injection site will be harvested and processed into paraffin sections and assessed by H & E staining and light microscopy. Tbese preliminary studies will quickly ascertain whether the new agents are associated with any unexpected severe toxicities, before proceeding to the more extensive and formal toxicity studies mandated by the regulatory agencies.

The following references are cited herein:

Adachi et al., (2001). Cancer Res. 61:7882-7888.

Blackwell et al., (2000). Hum Gene Ther. 11:1657-69.

Chartier et al., (1996). J. Virol. 70:4805-4810.

Defer et al., (1990). J. Virol. 64:3661-3673.

Dmitriev et al., (2000). J. Virol. 74:6875-6884.

Dmitriev et al., (1998). J. Virol. 72:9706-9713.

Douglas et al., (1999). 17:470-475.

Douglas et al., (1996). Nat Biotechnol. 14:1574-1578.

Durmort et al., (2001). Virology 285:302-312.

Fechner et al., (2000). Gene Ther. 7:1954-1968.

Gall et al., (1996). J. Virol. 70:2116-2123.

Kanerva et al., (2002). Clin. Cancer Res. 8:275-280.

Karayan et al., (1994). Virology 202:782-795.

Krasnykh et al., (2001). J. Virol. 75: 4176-4183.

Krasnykh et al., (1998). J. Virol. 72:1844-1852.

Krasnykh et al., (1996). J. Virol. 70:6839-6846.

Lindner et al., (1997). Biotechniques 22:140-149.

Maizel et al., (1968). Virology 36:115-125.

Michael et al., (1995). Gene Ther. 2:660-668.

Pereboev et al., (2001). J. Virol. 75:7107-7113.

Pickles et al., (1998). J. Virol. 72:6014-6023.

Roelvink et al., (1998). J. Virol. 72:7909-7915.

Russel, (2000). J. Gen. Virol. 81:2573-2604.

Rux and Burnett, (2000). Mol Ther. 1:18-30.

Seki et al., (2002). J. Virol. 76:1100-1108.

Shayakhmetov et al., (2000). J. Virol. 74:2567-2583.

Simons et al., (2000). Circulation 102:E73-86.

Stevenson et al., (1997). J. Virol. 71:4782-90.

Stevenson et al., (1995). J. Virol. 69:2850-2857.

Su et al., (2001). J. Vase. Res. 38:471-478.

Vigne et al., (1999). J Virol. 73:5156-61.

Von Seggern et al., (2000). J. Virol. 74:354-362.

Wickham et al., (1997). J. Virol. 71:8221-8229.

Wickham et al., (1996). Nat. Biotechnol. 14:1570-1573.

Xia et al., (2000). J. Virol. 74:11359-11366.

Yoshida et al., (1998). Hum. Gene Ther. 9:2503-2515.

Zabner et al., (1999). J. Virol. 73:8689-8695.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. An adenoviral vector comprising one or more heterologous targeting ligands incorporated into more than one capsid protein selected from the group consisting of hexon, knob domain of fiber protein, p3 protein, p9 protein and penton.

2. The adenoviral vector of claim 1, wherein said heterologous targeting ligand comprises the peptide sequence RGD.

3. The adenoviral vector of claim 1, wherein said heterologous targeting ligand is incorporated into the hexon and the knob domain of fiber protein.

4. The adenoviral vector of claim 1, wherein the length of the fiber shaft of said adenoviral vector is altered.

5. An adenoviral vector comprising one or more modified capsid protein selected from the group consisting of hexon, knob domain of fiber protein, p3 protein, p9 protein and penton, wherein said capsid proteins are modified by replacement with capsid proteins from another serotype.

6. The adenoviral vector of claim 5, wherein the fiber knob of adenovirus serotype 5 is replaced by the fiber knob of adenovirus serotype 3 or 37.

7. The adenoviral vector of claim 5, wherein the length of the fiber shaft of said adenoviral vector is altered.

8. The adenoviral vector of claim 5, further comprising a heterologous targeting ligand.

9. The adenoviral vector of claim 8, wherein the knob domain of fiber protein is replaced by knob domain from another serotype and the heterologous targeting ligand is incorporated in the knob domain.

10. The adenoviral vector of claim 9, wherein the fiber knob of adenovirus serotype 5 is replaced by the fiber knob of adenovirus serotype 3 or 37, and said heterologous targeting ligand comprises the peptide sequence RGD.

11. An adenoviral vector comprising capsid proteins derived from multiple distinct serotypes, wherein said capsid protein is selected from the group consisting of hexon, knob domain of fiber protein, p3 protein, p9 protein and penton

12. The adenoviral vector of claim 11, wherein said vector expresses fiber knob of adenovirus serotype 5 and adenovirus serotype 3.

13. An adenoviral vector which is charge-altered as a result of capsid modification, wherein said adenoviral vector also contains a modification selected from the group consisting of incorporating a heterologous targeting ligand, an altered fiber shaft length, and a capsid protein modified by replacement with capsid protein from another serotype.

14. The adenoviral vector of claim 13, wherein said capsid modification for charge alteration is selected from the group consisting of capsid addition, capsid deletion and capsid substitution.

15. The adenoviral vector of claim 13, wherein said capsid protein is selected from the group consisting of hexon, fiber protien, p3 protein, p9 protein and penton.

16. An adenoviral vector comprising at least one of the modifications selected from the group consisting of:
a) addition of a heterologous targeting ligand;
b) a fiber shaft with altered length;
c) capsid modification that results in charge alteration of said adenoviral vector; and
d) capsid protein modification by replacement with capsid protein from another serotype.

17. The adenoviral vector of claim 16, wherein said capsid protein is selected from the group consisting of hexon, fiber protein, p3 protein, p9 protein and penton.

18. The adenoviral vector of claim 16, wherein said capsid modification for charge alteration is selected from the group consisting of capsid addition, capsid deletion and capsid substitution.


SEQUENCE LISTING (9 sequences)

SEQ ID NO: 1 - DNA, 57 bp, Artificial Sequence
  mat_peptide: an added in coding sequence between the last coding codon of the chimeric Ad5/Ad3 fiber gene and its stop codon
ccatcagcctccgcatctgcttccgcccctagaggatcccatcaccatcaccatcac

SEQ ID NO: 2 - DNA, 21 bp, Artificial Sequence
  primer_bind: sense primer for Ad5 knob
agtgctcatcttattataaga

SEQ ID NO: 3 - DNA, 18 bp, Artificial Sequence
  primer_bind: sense primer for Ad3 knob
cgcacatcctatgttatg

SEQ ID NO: 4 - DNA, 20 bp, Artificial Sequence
  primer_bind: antisense primer for knob
caccaccgccctatcctgat

SEQ ID NO: 5 - DNA, 29 bp, Artificial Sequence
  primer_bind: sense primer for major late promoter (MLP)
ggttaattaagcatgtccctgactcgcat

SEQ ID NO: 6 - DNA, 26 bp, Artificial Sequence
  primer_bind: antisense primer for major late promoter (MLP)
ttgcgcgtgcacctggtgcccgacga

SEQ ID NO: 7 - DNA, 21 bp, Artificial Sequence
  primer_bind: primer for the sense strand of the Ad5 E4 region
tgacacgcatactcggagcta

SEQ ID NO: 8 - DNA, 20 bp, Artificial Sequence
  primer_bind: primer for the antisense strand of the E4 region
tttgagcagcaccttgcatt

SEQ ID NO: 9 - DNA, 21 bp, Artificial Sequence
  primer_bind: a TaqMan probe
cgccgcccatgcaacaagctt